(19) United States
(12) Patent Application Publication
(10) Pub. No.: US 2012/0202066 A1
(43) Pub. Date: Aug. 9, 2012

(54) Processes For Preparing Prasugrel And Pharmaceutically Acceptable Salts Thereof

(76) Inventors: Manne Satyanarayana Reddy, Hyderabad (IN); Srinivasan Thirumalai Rajan, Hyderabad (IN); Sajja Eswaraiah, Hyderabad (IN); Karamala Rama Subba Reddy, Hyderabad (IN); Bairy Kondal Reddy, Hyderabad (IN); Ghojala Venkat Reddy, Hyderabad (IN)

(21) Appl. No.: 13/500,846

(22) PCT Filed: Oct. 7, 2010
(86) PCT No.: PCT/IN2010/000665

§ 371 Date: Apr. 6, 2012

(30) Foreign Application Priority Data
Oct. 7, 2009 (IN) 2428/CHE/2009
Feb. 4, 2010 (IN) 278/CHE/2010
Jun. 2, 2010 (IN) 1515/CHE/2010

(51) Int. Cl. C07D 495/04; B65B 31/04; C07C 259/06; B01D 3/14; C07C 45/68; C07C 49/237

(52) U.S. Cl. 428/402; 546/114; 568/319; 568/329; 560/312; 203/73; 53/405

(57) ABSTRACT

Disclosed are improved processes for preparing prasugrel compound of formula-(1), its intermediates and pharmaceutically acceptable salts.

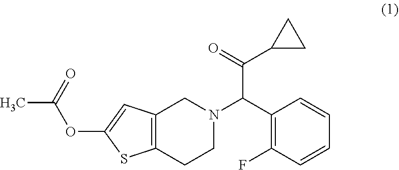

### RELATED APPLICATIONS

This application claims the benefit of priority of our Indian provisional application numbers: 2428/CHE/2009, filed on 7^{th} Oct. 2009, 278/CHE/2010, filed on 4^{th} Feb. 2010 and 1515/CHE/2010 filed on 2^{nd} Jun. 2010 which are incorporated herein by reference.

### FIELD OF THE INVENTION

The present invention relates to novel and improved processes for the preparation of prasugrel and its pharmaceutically acceptable salts, especially hydrochloride. Prasugrel hydrochloride is chemically known as 2-acetoxy-5-(α-cyclopropyl carbonyl-2-fluorobenzyl)-4,5,6,7-tetrahydrothieno[3,2-c]pyridine hydrochloride and having structural formula-1a,

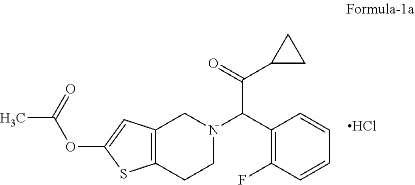

The present invention also relates to novel salts of 5-(α-cyclopropylcarbonyl-2-fluorobenzyl)-2-oxo-2,4,5,6,7,7a-hexahydro thieno[3,2-c] pyridine compound of formula-7 and its crystalline forms.

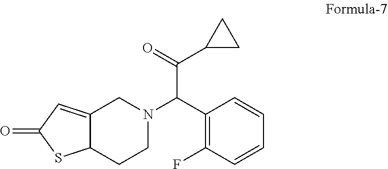

This invention also relates to a novel process for the preparation and purification of 1-cyclopropyl-2-(2-fluorophenyl)ethanone compound of formula-16.

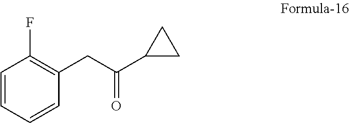

### BACKGROUND OF THE INVENTION

Prasugrel and its pharmaceutically acceptable salts have been disclosed in U.S. Pat. No. 5,288,726. The said patent also disclosed a process for the preparation of prasugrel and its pharmaceutically acceptable salts.

The disclosed process involves the preparation of Grignard reagent from 2-fluorobenzylbromide (i) then reaction with cyclopropylcyanide (ii) in ether to provide the compound (iii). The compound (iii) is brominated with N-bromosuccinamide (NBS) in the presence of dibenzoylperoxide provides α-cyclopropylcarbonyl-2-fluorobenzyl bromide (iv). Condensation of compound (iv) with 5,6,7,7a-tetrahydro-4H-thieno[3,2-c] pyridine-2-one (v) in presence of potassium carbonate in dimethylformamide provides 5-(α-cyclopropylcarbonyl-2-fluorobenzy)-2-oxo-2,4,5,6,7,7a-hexahydrothieno[3,2-c] pyridine (vi). Acetylation of 5-(α-cyclopropylcarbonyl-2-fluorobenzy)-2-oxo-2,4,5,6,7,7a-hexahydro thieno[3,2-c]pyridine(vi) using acetic anhydride in presence of sodium hydride provides prasugrel. As per the above process the compound (vi) obtained by the condensation of compound (iv) and (v) is very low in yield (32% only) which leads to increase in cost of the product. The yield and purity of the intermediates and final compounds are not satisfactory; also it involves the usage of column purification for isolation of intermediates as well as final compound. It involves the use of strong base like sodium hydride. Hence this process is difficult to perform in commercial scale.

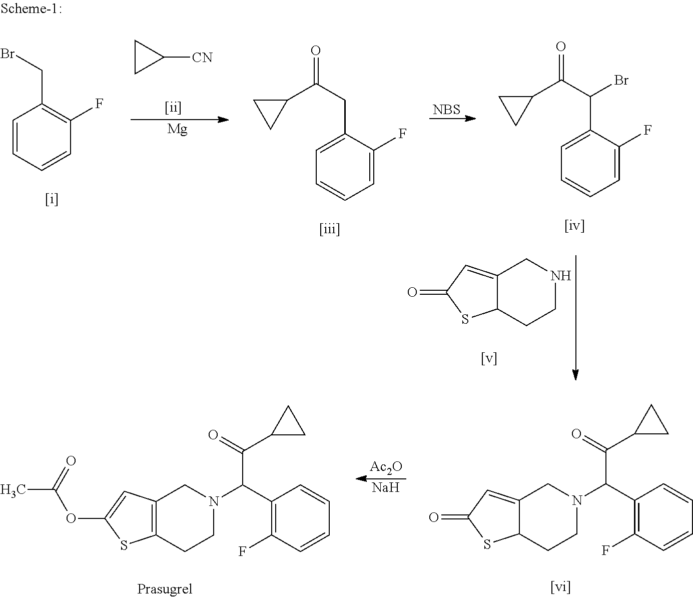

U.S. Pat. No. 5,288,726 also disclosed the hydrochloride salt of 5-(α-cyclopropylcarbonyl-2-fluorobenzyl)-2-oxo-2,4,5,6,7,7a-hexahydrothieno[3,2-c] pyridine with the melting point of 104-109° C., which is obtained by passing hydrogen chloride gas to a solution containing 5-(α-cyclopropylcarbonyl-2-fluorobenzyl)-2-oxo-2,4,5,6,7,7a-hexahydro thieno [3,2-c] pyridine in diethylether with a yield of 46%, which is very low.

The process for the preparation of 5,6,7,7a-tetrahydro-4H-thieno[3,2-c] pyridine-2-one(v) intermediate used in the above process is disclosed in U.S. Pat. No. 4,740,510. The disclosed process is schematically represented by the following scheme-2

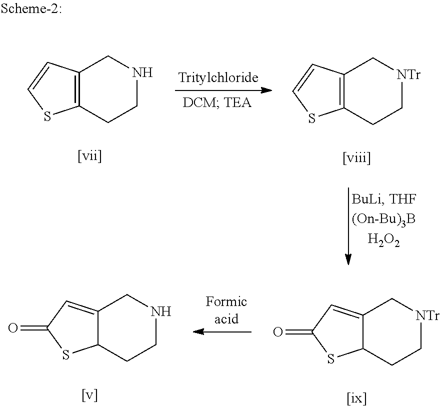

The disclosed process comprises of protecting the amino functional group of 4,5,6,7-tetrahydrothieno[3,2-c] pyridine compound (vii) by using triphenylmethylchloride which provides trityl protected 4,5,6,7-tetrahydrothieno[3,2-c] pyridine (viii) and converting it into 2-oxo derivative (ix) by treating with tri-n-butyl borate in presence of n-butyl lithium in tetrahydrofuran followed by treatment with hydrogen peroxide and finally deprotecting the trityl group using formic acid to provide 5,6,7,7a-tetrahydro-4H-thieno[3,2-c] pyridine-2-one (v). The said process involves unwanted protection and deprotection of amino group in order to introduce oxo group at second position of compound (vii), which leads to increase the number of steps, increased timeliness and cost of production.

Till the date, 5-(α-cyclopropylcarbonyl-2-fluorobenzyl)-2-oxo-2,4,5,6,7,7a-hexahydrothieno[3,2-c]pyridine was prepared from the condensation of 5,6,7,7a-tetrahydro-4H-thieno[3,2-c]pyridine-2-one or its salt with α-cyclopropylcarbonyl-2-fluorobenzyl bromide in presence of base and solvent with very low yields. Moreover the 5,6,7,7a-tetrahydro-4H-thieno[3,2-c] pyridine-2-one is less stable and hence its usage decreases the over all yield makes the process commercially not suitable. So there is a need in the art for novel process which avoids the use of less stable 5,6,7,7a-tetrahydro-4H-thieno[3,2-c] pyridine-2-one and which reduces the number of steps.

WO 2009/006859 disclosed a process for the preparation of prasugrel, which comprises of condensing the 3-cyclopropyl-1-(2-flurophenyl)-3-oxopropyl methane sulfonate with 2-oxo-thineotetrahydropyridine to provide 5-(α-cyclopropylcarbonyl-2-fluorobenzyl)-2-oxo-2,4,5,6,7-hexahydro thieno pyridine, which on acetylation with acetic anhydride provides prasugrel. This process involves the usage of column chromatography to get the pure product from the crude. Hence this process is not suitable for commercial scale.

Our International publication WO 2009/066326 disclosed a process for the preparation of prasugrel by acetylation of 5-(α-cyclopropylcarbonyl-2-fluorobenzyl)-2-oxo-2,4,5,6,7,7a-hexahydrothieno[3,2-c] pyridine with acetic anhydride in presence of triethyl amine using acetonitrile as a solvent. We surprisingly found that the usage of hydrocarbon solvent like toluene in place of acetonitrile provided excellent yield and purity. It is also having cost advantage over the usage of acetonitrile in the acetylation reaction. The said patent disclosed a powder X-ray diffractogram of prasugrel, the said crystalline form is similar to the prasugrel obtained as per the process disclosed in U.S. Pat. No. 5,288,726. The said crystalline form herein is designated as Form-I.

Polymorphism is the formation of a variety of crystalline forms of the same compound having distinct crystal structures and physical properties like melting points, X-ray diffraction pattern, infrared absorption pattern in fingerprint region, and solid state NMR spectrum. One crystalline form may give rise to thermal behavior different from that of another crystalline form. Different crystalline forms or polymorphs of the same pharmaceutical compounds can and reportedly do have different aqueous solubility. The difference in the physical properties of different crystalline forms results in some forms having distinct advantageous physical properties compared to other crystalline forms of the same compound. The discovery of new polymorphic forms of pharmaceutically useful compounds provides a new opportunity to improve the performance characteristics of a pharmaceutical product. Those skilled in the art understand that crystallization of an active pharmaceutical ingredient offers the best method for controlling important qualities like chemical quality, particle size, and polymorphic content. There is a need in the art for the preparation of new polymorphic forms of pharmaceutically acceptable salts of prasugrel as well as its intermediates and its salts.

1-cyclopropyl-2-(2-fluorophenyl)ethanone and its use in the preparation of prasugrel as well as process for its preparation was disclosed in U.S. Pat. No. 5,288,726. The disclosed process involves the reaction of 2-fluorobenzyl bromide with magnesium metal in diethyl ether followed by treatment with cyclopropyl cyanide to provide 1-cyclopropyl-2-(2-fluorophenyl)ethanone. Similar process is also disclosed in U.S. Pat. No. 6,693,115. The purity of the obtained compound is very low such as 50-55% by Gas chromatography. When the same has been used to proceed further without any purification in the preparation of prasugrel leads to the formation of corresponding impurities (i.e., impurities carried over from the impure material) which makes the process not suitable at commercial level.

International publication WO 2009/066326 disclosed an improved process for the preparation of 1-cyclopropyl-2-(2-flourophenyl)ethanone, which comprises of treating 2-fluorobenzyl bromide with magnesium metal in higher volumes of diethyl ether to provide 2-fluorobenzyl magnesium bromide, which on in-situ condensation with cyclopropyl cyanide in higher volumes of diethyl ether to get the 1-cyclopropyl-2-(2-fluorophenyl)ethanone. As per the said publication, higher volumes of diethyl ether is used to control the formation of 2-fluorobenzyl dimer impurity. The said patent does not describe any other impurity formation at this stage. Moreover the process involves excess volumes of ether solvent, which increase the cost of production. Hence the process may not be suitable at commercial level.

2-acetoxy-5-(α-methylcarbonyl-2-fluorobenzyl)-4,5,6,7-tetrahydrothieno[3,2-c] pyridine (herein designated as “methyl keto impurity”) having the following structural formula has been observed in prasugrel and its salts prepared by the processes known in the art

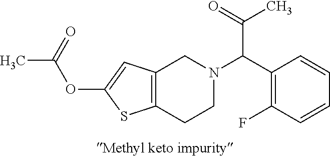

The said impurity has not been washed out by the conventional purification methods at final stages. Hence it is necessary to have a method to control the formation of the said impurity at origin.

We the present inventors working on prasugrel to find out the origin of the said impurity, after various experimentation we found that the origin of the impurity is at the formation of 1-methyl-2-(2-fluorophenyl)pethanone in the preparation of 1-cyclopropyl-2-(2-flourophenyl)ethanone i.e., during the reaction between 2-fluorobenzyl bromide with cyclopropyl cyanide under grignard condition. The said impurity is formed up to the maximum level of 4%. The said impurity carried further along with the keto compound in prior art process results in the formation of corresponding derivatives (i.e., 2-acetoxy-5-(α-methylcarbonyl-2-fluorobenzyl)-4,5,6,7-tetrahydrothieno[3,2-c] pyridine) in prasugrel. It is important for any pharmaceutical compound to be free of impurities or impurities to the level as per ICH guidelines. Hence it is necessary to develop a process which controls the formation of impurity at initial stages.

The formation of methyl keto impurity schematically represented by the following scheme

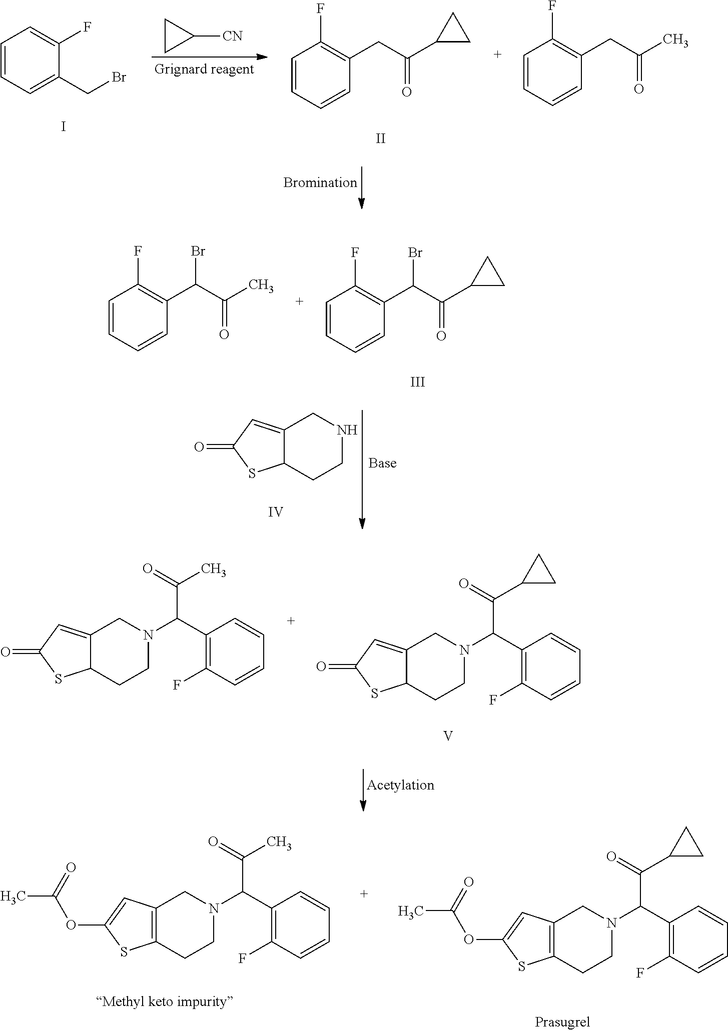

1-cyclopropyl-2-(2-flourophenyl)ethanone is a key intermediate in the preparation of pharmaceutically important compound such as prasugrel. It is more advantageous to have a novel process which provides a compound with high purity and yield and avoids the problems associated with the prior art.

Hence there is a need to develop a process which can be performed at an industrial scale. The present invention overcomes the problems associated with the prior art, and provides a process for the preparation of prasugrel and its pharmaceutically acceptable salts, with better yields and purity.

### BRIEF DESCRIPTION OF INVENTION

The present invention relates to a novel and improved processes for the preparation of Prasugrel, chemically known as 5-[(1RS)-2-cyclopropyl-1-(2-fluorophenyl)-2-oxoethyl]-4,5,6,7-tetrahydrothieno[3,2-c] pyridin-2-yl acetate compound of formula-1 and pharmaceutically acceptable salts thereof and its intermediates. It also relates to novel salts of 5-(α-cyclopropylcarbonyl-2-fluorobenzyl)-2-oxo-2,4,5,6,7,7a-hexahydrothieno[3,2-c] pyridine compound of formula-8 and its crystalline forms.

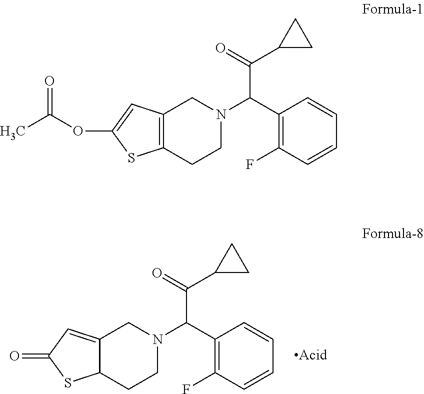

The first aspect of the present invention provides a novel process for the preparation of 5-[(1RS)-2-cyclopropyl-1-(2-fluorophenyl)-2-oxoethyl]-4,5,6,7-tetrahydrothieno[3,2-c] pyridin-2-yl acetate compound of formula-1 and pharmaceutically acceptable salts thereof, which comprises of,
a) Reacting the 4,5,6,7-tetrahydrothieno[3,2-c] pyridine compound of formula-2 or its salts, with α-cyclopropylcarbonyl-2-fluorobenzyl bromide compound of formula-3, in presence of a suitable base in a suitable solvent to provide 5-(α-cyclopropylcarbonyl-2-fluorobenzyl)-4,5,6,7,-tetrahydrothieno[3,2-c] pyridine compound of formula-4,
b) converting the 5-(α-cyclopropylcarbonyl-2-fluorobenzyl)-4,5,6,7,-tetrahydrothieno [3,2-c]pyridine compound of formula-4 into 5-(α-cyclopropylcarbonyl-2-fluorobenzyl)-2-oxo-2,4,5,6,7,7a-hexahydrothieno[3,2-c]pyridine compound of Formula-7, by in-situ protecting the keto functionality of compound of formula-4 as an enolate by treating with a lithium reagent and by introducing a boronic group —B(OR′)₂ at second position of thieno[3,2-c] pyridine skeleton by treating it with second lithium reagent in a suitable solvent and a suitable boronating agent, in presence or absence of co-solvent and subsequent oxidation by treating it with suitable oxidizing agent to provide the compound of formula-7,
c) acetylating the compound of Formula-7 with a suitable acetylating agent like acetic anhydride in a suitable solvent in presence of a suitable organic base selected to provide the compound of formula-1,
d) optionally converting the prasugrel into its acid addition salts by treating it with a suitable acid in a suitable solvent to provide an acid addition salt of prasugrel.

The second aspect of the present invention is to provide a novel process for the preparation of 5-(α-cyclopropylcarbonyl-2-fluorobenzyl)-2-oxo-2,4,5,6,7,7a-hexahydrothieno[3,2-c]pyridine compound of Formula-7, which comprises of,
a) Reacting the 4,5,6,7-tetrahydrothieno[3,2-c] pyridine compound of formula-2 or its salts, with α-cyclopropylcarbonyl-2-fluorobenzyl bromide compound of formula-3, in presence of a suitable base in a suitable solvent to provide 5-(α.-cyclopropyl carbonyl-2-fluorobenzyl)-4,5,6,7-tetrahydrothieno[3,2-c]pyridine compound of formula-4,
b) converting the 5-(α-cyclopropylcarbonyl-2-fluorobenzyl)-4,5,6,7,-tetrahydrothieno [3,2-c]pyridine compound of formula-4 into 5-(α-cyclopropylcarbonyl-2-fluorobenzyl)-2-oxo-2,4,5,6,7,7a-hexahydrothieno[3,2-c]pyridine compound of Formula-7, by in-situ protecting the keto functionality of compound of formula-4 as an enolate by treating with a lithium reagent and by introducing a boronic group —B(OR′)₂ at second position of thieno[3,2-c] pyridine skeleton by treating it with second lithium reagent in a suitable solvent and a suitable boronating agent, in presence or absence of co-solvent and subsequent oxidation by treating it with suitable oxidizing agent to provide the compound of formula-7.

The third aspect of the present invention relates to acid addition salts of 5-(α-cyclopropylcarbonyl-2-fluorobenzyl)-4,5,6,7,-tetrahydrothieno [3,2-c] pyridine compounds of general Formula-9 and process for their preparation as well as their use.

The fourth aspect of the present invention is to provide the novel salts of 5-(α-cyclopropylcarbonyl-2-fluorobenzyl)-2-oxo-2,4,5,6,7,7a-hexahydro thieno[3,2-c] pyridine with the proviso that the salt is not a hydrochloride.

The fifth aspect of the present invention is to provide a process for the preparation of novel salts of 5-(α-cyclopropylcarbonyl-2-fluorobenzyl)-2-oxo-2,4,5,6,7,7a-hexahydro thieno[3,2-c] pyridine.

The sixth aspect of the present invention is to provide a crystalline form of 5-(α-cyclopropylcarbonyl-2-fluorobenzyl)-2-oxo-2,4,5,6,7,7a-hexahydrothieno[3,2-c] pyridine hydrobromide as well as a process for its preparation. The novel crystalline form of the present invention is characterized by its PXRD, IR spectrum and DSC thermogram, substantially as shown in FIG. 1, 2 & 3 respectively.

The seventh aspect of the present invention is to provide the use of novel salts of 5-(α-cyclopropylcarbonyl-2-fluorobenzyl)-2-oxo-2,4,5,6,7,7a-hexahydrothieno[3,2-c] pyridine of the present invention and crystalline form of 5-(α-cyclopropylcarbonyl-2-fluorobenzyl)-2-oxo-2,4,5,6,7,7a-hexahydrothieno[3,2-c] pyridine hydrobromide in the preparation of highly pure 5-(α-cyclopropylcarbonyl-2-fluorobenzyl)-2-oxo-2,4,5,6,7,7a-hexahydro thieno[3,2-c] pyridine as well as the usage in the preparation of highly pure prasugrel and its pharmaceutically acceptable salts.

The eighth aspect of the present invention is to provide a novel crystalline form of 5-(α-cyclopropylcarbonyl-2-fluorobenzyl)-2-oxo-2,4,5,6,7,7a-hexahydrothieno[3,2-c] pyridine as well as process for its preparation.

The ninth aspect of the present invention is to provide a novel crystalline form-N of prasugrel free base as well as a process for its preparation. The novel crystalline form-N of prasugrel is characterized by its Powder X-ray diffractogram and is shown in FIG. 5.

The tenth aspect of the present invention is to provide an improved process for the preparation of prasugrel compound of formula-1 and its pharmaceutically acceptable salts, which comprises of acetylating the 5-(α-cyclopropylcarbonyl-2-fluorobenzyl)-2-oxo-2,4,5,6,7,7a-hexahydrothieno[3,2-c] pyridine with a suitable acetylating agent in presence of a suitable organic base in a suitable hydrocarbon solvent, followed by crystallization from a suitable solvent to provide prasugrel compound of formula-1.

The eleventh aspect of the present invention provides an improved and one-pot process for the preparation of prasugrel and its pharmaceutically acceptable salts.

The twelfth aspect of the present invention is to provide a novel process for the preparation of highly pure 1-cyclopropyl-2-(2-fluorophenyl)ethanone compound of formula-16, which comprises of the following steps;
1. Reacting the 2-(2-fluorophenyl)acetic acid compound of formula-13 with N,O-dialkylhydroxylamine or its salts compound of general formula-14 in presence of a base in a suitable solvent provides 2-(2-fluorophenyl)-N-alkoxy-N-alkylacetamide compound of general formula-15,
2. reacting the compound of general formula-15 with cyclopropyl bromide in presence of Grignard reagent and in presence or absence of a catalyst in a suitable solvent provides pure 1-cyclopropyl-2-(2-fluorophenyl)pethanone compound of formula-16.

Further the present invention also provides novel 2-(2-fluorophenyl)-N-alkoxy-N-alkylacetamide compound of general formula-15 and its use.

The thirteenth aspect of the present invention is to provide a process for the preparation of highly pure prasugrel compound of formula-1 and its pharmaceutically acceptable salts, which comprises of preparing the compound of formula-16 as per the twelfth aspect of the present invention and converting the same into prasugrel compound of formula-1 by the conventional methods known in the art.

The fourteenth aspect of the present invention is to provide a process for the purification of 1-cyclopropyl-2-(2-fluorophenyl)ethanone compound of formula-16 or process for the removing of 1-methyl-2-(2-fluorophenyl)ethanone from compound of formula-16, which comprises of subjecting the crude compound of formula-16 to high vacuum distillation/fractional distillation. Collecting the required product by fractionation at their specific boiling point.

The fifteenth aspect of the present invention is to provide a highly pure 1-cyclopropyl-2-(2-fluorophenyl)ethanone compound of formula-16 having 5.0% or less of 1-methyl-2-(2-fluorophenyl)ethanone by GC.

The sixteenth aspect of the present invention is to provide highly pure 5-(2-cyclopropyl-1-(2-fluorophenyl)-2-oxoethyl)-4,5,6,7-tetrahydrothieno[3,2-c]pyridin-2-yl acetate compound of formula-1 and its pharmaceutically acceptable salts having 2-acetoxy-5-(α-methylcarbonyl-2-fluorobenzyl)-4,5,6,7-tetrahydrothieno [3,2-c] pyridine in the level of less than 4.0% by HPLC.

The seventeenth aspect of the present invention is to provide a process for the purification of the prasugrel using suitable solvent to get pure prasugrel compound of formula-1.

### BRIEF DESCRIPTION OF DRAWINGS

FIG. 1: Illustrates the powder X-ray diffractogram of crystalline form-M of 5-(α-cyclopropylcarbonyl-2-fluorobenzyl)-2-oxo-2,4,5,6,7,7a-hexahydrothieno[3,2-c] pyridine hydrobromide.

FIG. 2: Illustrates the IR spectrum of crystalline form-M of 5-(α-cyclopropylcarbonyl-2-fluorobenzyl)-2-oxo-2,4,5,6,7,7a-hexahydrothieno[3,2-c] pyridine hydrobromide.

FIG. 3: Illustrates the DSC thermo gram of crystalline form-M of 5-(α-cyclopropylcarbonyl-2-fluorobenzyl)-2-oxo-2,4,5,6,7,7a-hexahydrothieno[3,2-c] pyridine hydrobromide

FIG. 4: Illustrates the powder X-ray diffractogram of crystalline form-S of 5-(α-cyclopropylcarbonyl-2-fluorobenzyl)-2-oxo-2,4,5,6,7,7a-hexahydrothieno[3,2-c] pyridine

FIG. 5: Illustrates the powder X-ray diffractogram of crystalline form-N of Prasugrel

### DETAILED DESCRIPTION OF THE INVENTION

As used herein, the term “PG” refers to protecting group which is selected from trityl, BOC (tert-butyloxy carbonyl) and benzoyl.

As used herein, the term “pharmaceutically acceptable salts” refers to the acid addition salt compound formed with a suitable acid selected from an inorganic acid such as hydrochloric acid, hydrobromic acid; or an organic acid such as benzene sulfonic acid, maleic acid, oxalic acid, fumaric acid, succinic acid, p-toluenesulfonic acid and malic acid.

As used herein, the term “highly pure prasugrel” refers to prasugrel with the purity equal to 99.50% or more by HPLC.

As used herein, the term “highly pure 1-cyclopropyl-2-(2-fluorophenyl) ethanone” refers to 1-cyclopropyl-2-(2-fluorophenyl)ethanone with the purity equal to 85.00% or more by HPLC.

As used here in the term “alcoholic solvents” refers to methanol, ethanol, isopropanol, n-propanol, butanol and the like; the term “ester solvents” refers to ethyl acetate, methyl acetate, n-butyl acetate, isobutyl acetate, sec-butyl acetate, isopropyl acetate and the like; the term “ether solvents” refers to tetrahydrofuran, diethyl ether, methyl tert-butyl ether and the like; the term “ketone solvents” refers to acetone, methyl ethyl ketone, methyl isobutyl ketone and the like; the term “hydrocarbon solvents” refers to toluene, xylene, cyclohexane, hexane, heptane and the like; the term “chloro solvents” refers to methylene chloride, ethylene dichloride, carbon tetra chloride, chloroform and the like; polar aprotic solvents refers to dimethylformamide, dimethylacetamide, dimethyl sulfoxide, tetrahydrofuran and the like; the term “nitrile solvents” refers to acetonitrile and the like; the term “polar solvent” refers to water and the like and mixtures thereof.

As used herein the term “inorganic base” refers to alkali metal carbonates like sodium carbonate, potassium carbonate; alkali metal hydroxide like sodium hydroxide, potassium hydroxide; alkali metal bicarbonates like sodium bicarbonate, potassium bicarbonate; alkali alkoxides like sodium methoxide, sodium tertiary butoxide, potassium tertiary butoxide and the like; the term “organic base” refers to triethylamine, isopropyl ethylamine, diisopropyl amine, diisopropylethyl amine tributyl amine, pyridine, 4-dimethylaminopyridine, N-methylmorpholine and piperidine, pyridine and the like.

As used herein the term “highly pure” refers to the purity of the compound, in which the compound has the purity of about 96.00% or more by HPLC, preferably greater than 99.00% and more preferably greater than 99.90% by HPLC.

As used here in the term “crude” refers to the compound obtained directly after the reaction may be in the form of solid, residue or oily residue or the compound before purification.

As used herein the term “highly pure” refers to the purity of the compound, in which the compound has the purity of about 96.00% or more by HPLC, preferably greater than 99.00% and more preferably greater than 99.90% by HPLC.

The first aspect of the present invention provides a novel process for the preparation of 5-[(1RS)-2-cyclopropyl-1-(2-fluorophenyl)-2-oxoethyl]-4,5,6,7-tetrahydro thieno[3,2-c]pyridin-2-yl acetate compound of formula-1

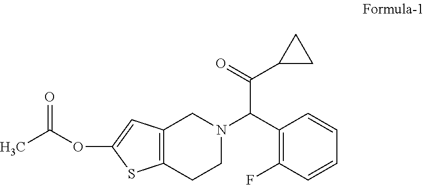

and pharmaceutically acceptable salts thereof, which comprises of
a) Reacting the 4,5,6,7-tetrahydrothieno[3,2-c] pyridine compound of formula-2 or its salts,

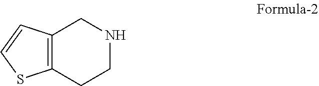

with α-cyclopropylcarbonyl-2-fluorobenzyl bromide, compound of formula-3,

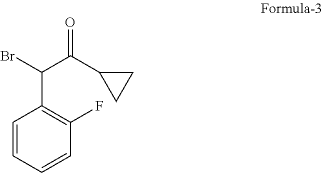

in presence of a suitable base in a suitable solvent to provide 5-(α-cyclopropylcarbonyl-2-fluorobenzyl)-4,5,6,7,-tetrahydrothieno[3,2-c] pyridine compound of formula-4,

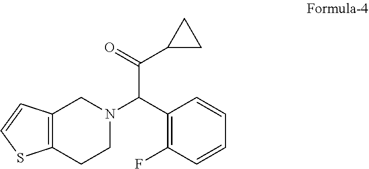

b) converting the 5-(α-cyclopropylcarbonyl-2-fluorobenzyl)-4,5,6,7,-tetrahydrothieno [3,2-c]pyridine compound of formula-4 into 5-(α-cyclopropylcarbonyl-2-fluorobenzyl)-2-oxo-2,4,5,6,7,7a-hexahydrothieno[3,2-c]pyridine compound of Formula-7, by in-situ protecting the keto functionality of compound of formula-4 as an enolate by treating with a lithium reagent and by introducing a boronic group —B(OR′)₂ at second position of thieno [3,2-c] pyridine skeleton by treating it with second lithium reagent in a suitable solvent and a suitable boronating agent, in presence or absence of co-solvent and subsequent oxidation by treating it with suitable oxidizing agent to provide compound of Formula-7,

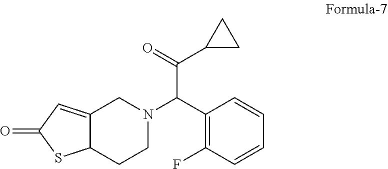

c) acetylating the compound of Formula-7 with a suitable acetylating agent in a suitable solvent in presence of a suitable organic base to provide the compound of formula-1,
d) optionally converting the prasugrel into its acid addition salts by treating it with a suitable acid in a suitable solvent to provide an acid addition salt of prasugrel.

Wherein in step a) the suitable base is selected from a group consisting of alkali metal carbonates like sodium carbonate, potassium, carbonate; or an alkali metal hydroxide like sodium hydroxide, potassium hydroxide; or alkali metal bicarbonates like sodium bicarbonate, potassium bicarbonate; alkali metal alkoxides like sodium tertiary butoxide, potassium tertiary butoxide or an organic base like triethylamine, tributylamine, diisopropylethlyamine preferably potassium carbonate, in a suitable solvent selected from aliphatic hydrocarbons like hexane, cyclohexane, petroleum ether; or aromatic hydrocarbons like xylene, toluene; or halogenated hydrocarbons like dichloromethane, chloroform, 1,2-dichloroethane; or ethers like diethyl ether, diisopropyl ether, tetrahydrofuran, dimethoxy ethane; or ketones like acetone, methyl ethyl ketone, diethyl ketone; or acetates like ethyl acetate, propyl acetate, butyl acetate; alcohols like methanol, ethanol, propanol, butanol, isopropanol; or nitriles like acetonitrile and propionitrile; dimethyl formamide, dimethyl acetamide and dimethyl sulfoxide or mixtures thereof preferably acetonitrile;

In step b) the suitable lithium derivative for protecting the keto functionality as enolate is selected from n-butyl lithium, sec-butyl lithium, tert-butyl lithium, lithium hexamethyldisilazide and lithium diisopropylamide preferably lithium diisopropylamide; the suitable boronating agent is selected from boron oxides such as B₂O₃, boron acids such as H₃BO₃, lower alkyl esters of boron acids such as trimethylborate, triethylborate, tri n-butylborate, boron halides like BF₃, BCl₃, salts of boron acids like sodium borate, ammonium borate preferably tri n-butylborate; the suitable lithiating agent for the generation of lithium salt at 2^{nd} position of thieno[3,2-c] pyridine skeleton is selected from n-butyl lithium, sec-butyl lithium, tert-butyl lithium lithium hexamethyldisilazide and lithium diisopropylamide; preferably n-butyl lithium; the suitable oxidising agent is selected from nitric acid, hydrogen peroxide, per acids such as peracetic acid, trifluoro peracetic acid, perbenzoic acid, m-chloro perbenzoic acid and the like; ozone, manganese dioxide, potassium permanganate, chromic acid, chromium trioxide, selenium dioxide, sodium hypochlorite, sodium metaperiodate and the like, preferably hydrogen peroxide; the suitable co-solvent is selected from tetramethyl urea(TMU), 1,3-Dimethyl-3,4,5,6-tetrahydro-2(1H)-pyrimidinone (DMPU), N-Methyl-2-pyrrolidone (NMP), hexamethylphosphoramide (HMPA) and the like; and the suitable solvent is selected from aliphatic hydrocarbons like hexane, cyclohexane, petroleum ether; or aromatic hydrocarbons like xylene, toluene; or halogenated hydrocarbons like dichloromethane, chloroform, 1,2-dichloroethane; or ethers like diethyl ether, diisopropyl ether, tetrahydrofuran, dimethoxy ethane; or ketone solvents or acetate solvents; alcohol solvents or nitrite solvents like acetonitrile and propionitrile; dimethyl formamide, dimethyl acetamide and dimethyl sulfoxide or mixtures thereof preferably tetrahydrofuran. Optionally in place of second lithium reagent dialkyl zinc compounds like diethyl zinc, dimethyl zinc ethyl methyl zinc and the like also can be used.

in step c) the suitable acetylating agent is like acetic anhydride in a suitable solvent selected from diethylether, tetrahydrofuran, dioxane, acetone, methylethyl ketone, ethyl acetate, acetonitrile, dimethyl formamide, dimethyl acetamide and dimethyl sulfoxide preferably acetonitrile, in presence of a suitable organic base selected from triethyl amine, tributyl amine, pyridine, 4-dimethylaminopyridine, N-methylmorpholine and diisopropylethyl amine preferably triethylamine,

in step d) the suitable acid selected from an inorganic acids such as hydrochloric acid, hydrobromic acid; or an organic acids such as benzene sulfonic acid, maleic acid, oxalic acid, fumaric acid, succinic acid, p-toluenesulfonic acid and malic acid, in a suitable solvent selected from aliphatic hydrocarbons like hexane, cyclohexane, petroleum ether; or aromatic hydrocarbons like xylene, toluene; or halogenated hydrocarbons like dichloromethane, chloroform, 1,2-dichloroethane; or ethers like diethyl ether, diisopropyl ether, tetrahydrofuran, dimethoxy ethane; or ketones like acetone, methyl ethyl ketone, diethyl ketone; or acetates like ethyl acetate, propyl acetate, butyl acetate; alcohol like isopropyl alcohol; or nitriles like acetonitrile and propionitrile or mixtures thereof.

The present invention was schematically represented as follows.

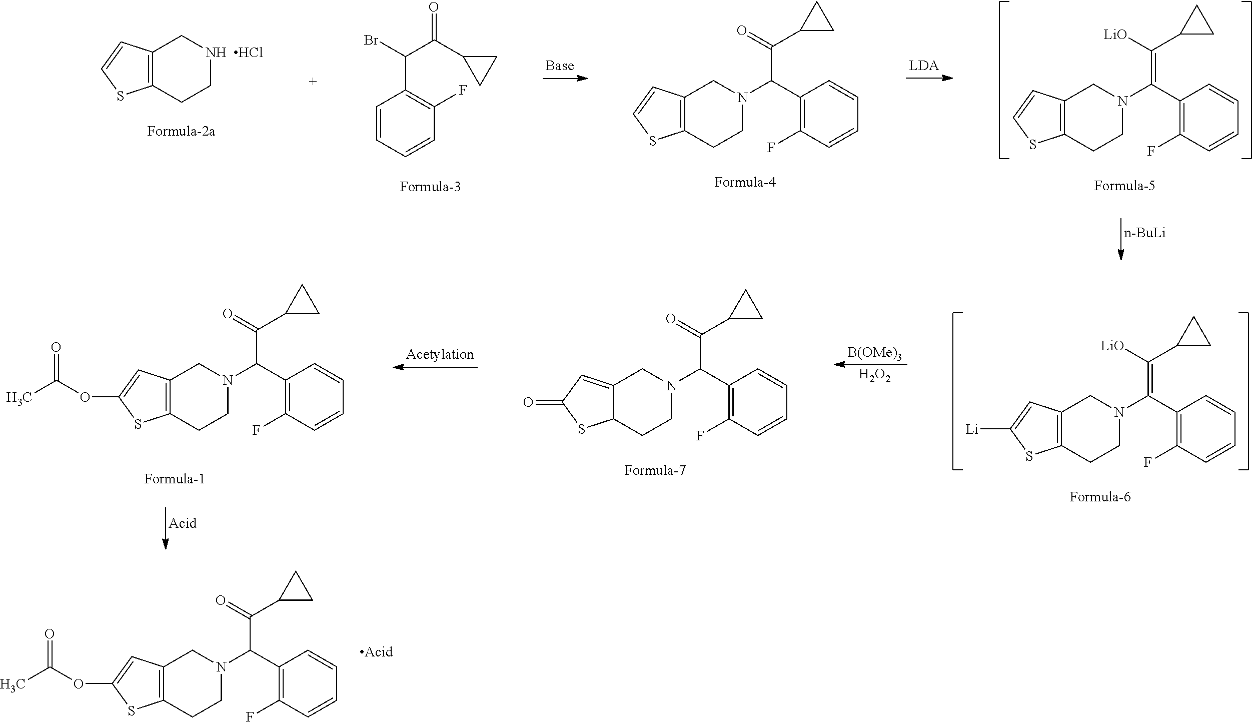

The second aspect of the present invention provides a process for the preparation of 5-(α-cyclopropylcarbonyl-2-fluorobenzyl)-2-oxo-2,4,5,6,7,7a-hexahydrothieno[3,2-c] pyridine compound of Formula-7,

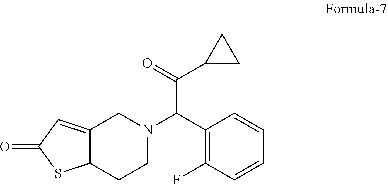

Which comprises of;
a) Reacting the 4,5,6,7-tetrahydrothieno[3,2-c] pyridine compound of formula-2 or its salts,

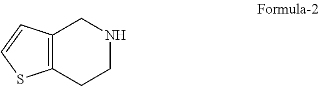

with α-cyclopropylcarbonyl-2-fluorobenzyl bromide compound of formula-3,

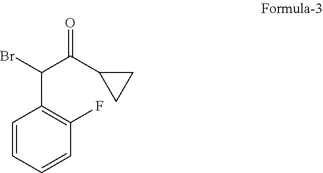

in presence of a suitable base in a suitable solvent to provide 5-(α-cyclopropylcarbonyl-2-fluorobenzyl)-4,5,6,7,-tetrahydrothieno[3,2-c] pyridine compound of formula-4,

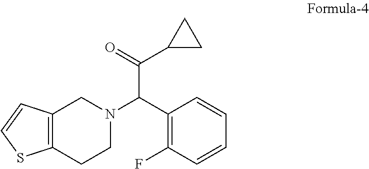

b) converting the 5-(α-cyclopropylcarbonyl-2-fluorobenzyl)-4,5,6,7,-tetrahydrothieno [3,2-c]pyridine compound of formula-4 into 5-(α-cyclopropylcarbonyl-2-fluorobenzyl)-2-oxo-2,4,5,6,7,7a-hexahydrothieno[3,2-c]pyridine compound of Formula-7, by in-situ protecting the keto functionality of compound of formula-4 as an enolate by treating with a lithium reagent and by introducing a boronic group —B(OR′)₂ at second position of thieno[3,2-c] pyridine skeleton by treating it with second lithium reagent in a suitable solvent and a suitable boronating agent, in presence or absence of co-solvent and subsequent oxidation by treating it with suitable oxidizing agent to provide compound of Formula-7,

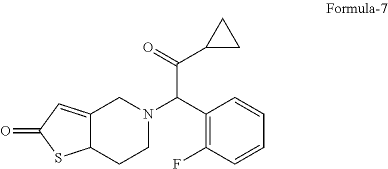

Wherein in step a) the suitable base is selected from a group consisting of alkali metal carbonates, or an alkali metal hydroxides, or alkali metal bicarbonates alkali metal alkoxides or an organic base like triethylamine, tributylamine, diisopropylethlyamine preferably potassium carbonate, in a suitable solvent selected from aliphatic hydrocarbons like hexane, cyclohexane, petroleum ether; or aromatic hydrocarbons like xylene, toluene; or halogenated hydrocarbons like dichloromethane, chloroform, 1,2-dichloroethane; or ethers like diethyl ether, diisopropyl ether, tetrahydrofuran, dimethoxy ethane or ketone solvents or ester solvents or alcohol solvents or nitriles like acetonitrile and propionitrile; dimethyl formamide, dimethyl acetamide and dimethyl sulfoxide or mixtures thereof preferably acetonitrile;

In step b) the suitable lithium reagent for protecting the keto functionality as enolate is selected from n-butyl lithium, sec-butyl lithium, tert-butyl lithium lithium hexamethyldisilazide and lithium diisopropylamide preferably lithium diisopropylamide; the suitable boronating agent is selected from boron oxides such as B₂O₃, boron acids such as H₃BO₃, lower alkyl esters of boron acids such as trimethylborate, triethylborate, tri n-butylborate, boron halides like BF₃, BCl₃, salts of boron acids like sodium borate, ammonium borate preferably tri n-butylborate; the suitable second lithiating agent for the generation of lithium salt at 2^{nd} position of thieno[3,2-c] pyridine skeleton is selected from n-butyl lithium, sec-butyl lithium, tert-butyl lithium, lithium hexamethyldisilazide and lithium diisopropylamide; preferably n-butyl lithium; and the suitable oxidising agent is selected from nitric acid, hydrogen peroxide, per acids such as peracetic acid, trifluoro peracetic acid, perbenzoic acid, m-chloro perbenzoic acid and the like; ozone, manganese dioxide, potassium permanganate, chromic acid, chromium trioxide, selenium dioxide, sodium hypochlorite, sodium metaperiodate and the like, preferably hydrogen peroxide; the suitable co solvent is selected from tetramethyl urea(TMU), 1,3-Dimethyl-3,4,5,6-tetrahydro-2(1H)-pyrimidinone (DMPU), N-Methyl-2-pyrrolidone (NMP), hexamethylphosphoramide (HMPA) and the like; and the suitable solvent is selected from aliphatic hydrocarbons like hexane, cyclohexane, petroleum ether; or aromatic hydrocarbons like xylene, toluene; or halogenated hydrocarbons like dichloromethane, chloroform, 1,2-dichloroethane; or ethers like diethyl ether, diisopropyl ether, tetrahydrofuran, dimethoxy ethane; or ketones like acetone, methyl ethyl ketone, diethyl ketone; or acetates like ethyl acetate, propyl acetate, butyl acetate; alcohols like methanol, ethanol, propanol, butanol, isopropanol; or nitriles like acetonitrile and propionitrile; dimethyl formamide, dimethyl acetamide and dimethyl sulfoxide or mixtures thereof preferably tetrahydrofuran. Optionally in place of second lithium reagent dialkyl zinc compounds like diethyl zinc, dimethyl zinc ethyl methyl zinc and the like also can be used.

In the above aspect of the present invention the conversion of the 5-(α-cyclopropylcarbonyl-2-fluorobenzyl)-4,5,6,7,-tetrahydrothieno [3,2-c]pyridine compound of formula-4 into 5-(α-cyclopropylcarbonyl-2-fluorobenzyl)-2-oxo-2,4,5,6,7,7a-hexahydro thieno[3,2-c]pyridine compound of Formula-7 can be carried out using a single lithium reagent for both enolation as well as formation of lithium salt during the introduction of a boronic group —B(OR′)₂ at second position of thieno[3,2-c] pyridine skeleton.

The third aspect of the present invention provides novel acid addition salts of 5-(α-cyclopropylcarbonyl-2-fluorobenzyl)-4,5,6,7,-tetrahydrothieno[3,2-c]pyridine compound represented by the following structural formula-9.

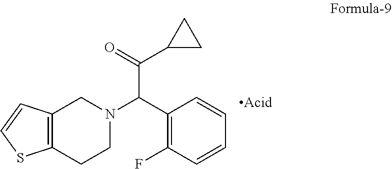

Wherein the acid is a acid group which is capable of forming addition salts with the compound of formula-4 and such acid is selected from inorganic acids such as hydrobromic acid, sulfuric acid, nitric acid or organic acids such as benzene sulfonic acid, maleic acid, oxalic acid, fumaric acid, succinic acid, p-tolunesulfonic acid and malic acid, provided that the acid is not hydrochloric acid.

The process for the preparation of acid addition salts of 5-(α-cyclopropylcarbonyl-2-fluorobenzyl)-4,5,6,7,-tetrahydrothieno[3,2-c]pyridine compounds of general Formula-8, comprises of treating the 5-(α-cyclopropylcarbonyl-2-fluorobenzyl)-4,5,6,7,-tetrahydrothieno[3,2-c] pyridine compound of formula-4 with a suitable acid in a suitable solvent, to provide the corresponding salts compounds of general Formula-9.

The suitable acid used is selected from an inorganic acids such as hydrochloric acid, hydrobromic acid, sulfuric acid, nitric acid; or an organic acid such as benzene sulfonic acid, maleic acid, oxalic acid, fumaric acid, succinic acid, p-tolunesulfonic acid and malic acid; and the suitable solvent is selected from aliphatic hydrocarbons like hexane, cyclohexane, petroleum ether; or aromatic hydrocarbons like xylene, toluene; or halogenated hydrocarbons like dichloromethane, chloroform, 1,2-dichloroethane; or ethers like diethyl ether, diisopropyl ether, tetrahydrofuran, dimethoxy ethane; or ketones like acetone, methyl ethyl ketone, diethyl ketone; or acetates like ethyl acetate, propyl acetate, butyl acetate; alcohols like methanol, ethanol, propanol, butanol, isopropanol; or nitriles like acetonitrile and propionitrile; dimethyl formamide, dimethyl acetamide and dimethyl sulfoxide or mixtures thereof; the suitable temperature is −20° C. to reflux temperature of the solvent used.

The acid addition salts compound of general Formula-9 of the present invention is used to prepare highly pure compound of Formula-7 and prasugrel or its pharmaceutically acceptable salt.

As used herein the present invention the term “highly pure” refers to the compound with purity greater than 99.00% by HPLC, preferably >99.50% by HPLC and more preferably >99.90% by HPLC.

The 5,6,7,7a-tetrahydro-4H-thieno[3,2-c] pyridine-2-[4H]-one compound of formula-17 can also be prepared by treating N-trityl 4,5,6,7-tetrahydrothieno[3,2-c]pyridine compound of formula-10 with dialkyl zinc compounds like diethyl zinc, dimethyl zinc ethyl methyl zinc etc. followed by treating with suitable boronic agent and subsequent treatment with a suitable oxidizing agent to provide N-trityl 5,6,7,7a-tetrahydro-4H-thieno[3,2-c] pyridine-2-one compound of formula-11 which is treated with formic acid to provide compound-17. The suitable boronating agent and suitable oxidizing agents are same as described above.

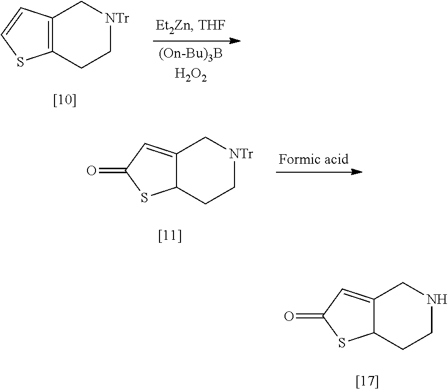

The fourth aspect of the present invention provides the novel salts of 5-(α-cyclopropylcarbonyl-2-fluorobenzyl)-2-oxo-2,4,5,6,7,7a-hexahydrothieno[3,2-c] pyridine and is represented by the following general formula-8,

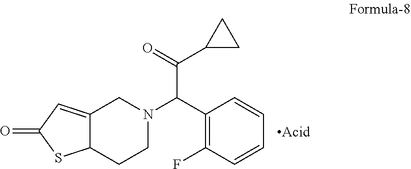

wherein “Acid” is an acid which is capable of forming acid addition salt with 5-(α-cyclopropylcarbonyl-2-fluorobenzyl)-2-oxo-2,4,5,6,7,7a-hexahydrothieno[3,2-c] pyridine and is selected from group comprising of oxalic acid, succinic acid, fumaric acid, malonic acid, malic acid, maleic acid, d-tartaric acid, 1-tartaric acid, dl-tartaric acid, citric acid, methanesulfonic acid, paratoluene sulfonic acid, acetic acid, sulfuric acid, phosphoric acid or hydrobromic acid, with a proviso that the acid is not hydrochloric acid.

The fifth aspect of the present invention provides a process for the preparation of novel salts of 5-(α-cyclopropylcarbonyl-2-fluorobenzyl)-2-oxo-2,4,5,6,7,7a-hexahydrothieno[3,2-c] pyridine, which comprises of treating the 5-(α-cyclopropylcarbonyl-2-fluorobenzyl)-2-oxo-2,4,5,6,7,7a-hexahydrothieno[3,2-c] pyridine with a suitable acid selected from the acids which are defined above, in a suitable solvent selected from alcohols, ketones, esters, hydrocarbons, polar aprotic solvents, polar solvents, chloro solvents, nitriles or mixtures thereof for the sufficient period of time, to provide the corresponding salt of 5-(α-cyclopropylcarbonyl-2-fluorobenzyl)-2-oxo-2,4,5,6,7,7a-hexahydro thieno[3,2-c] pyridine.

The sixth aspect of the present invention provides a crystalline form of 5-(α-cyclopropylcarbonyl-2-fluorobenzyl)-2-oxo-2,4,5,6,7,7a-hexahydro thieno[3,2-c] pyridine hydrobromide compound of formula-8a.

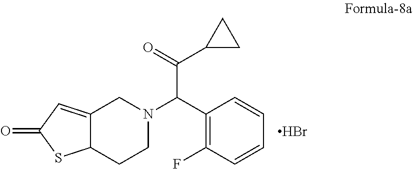

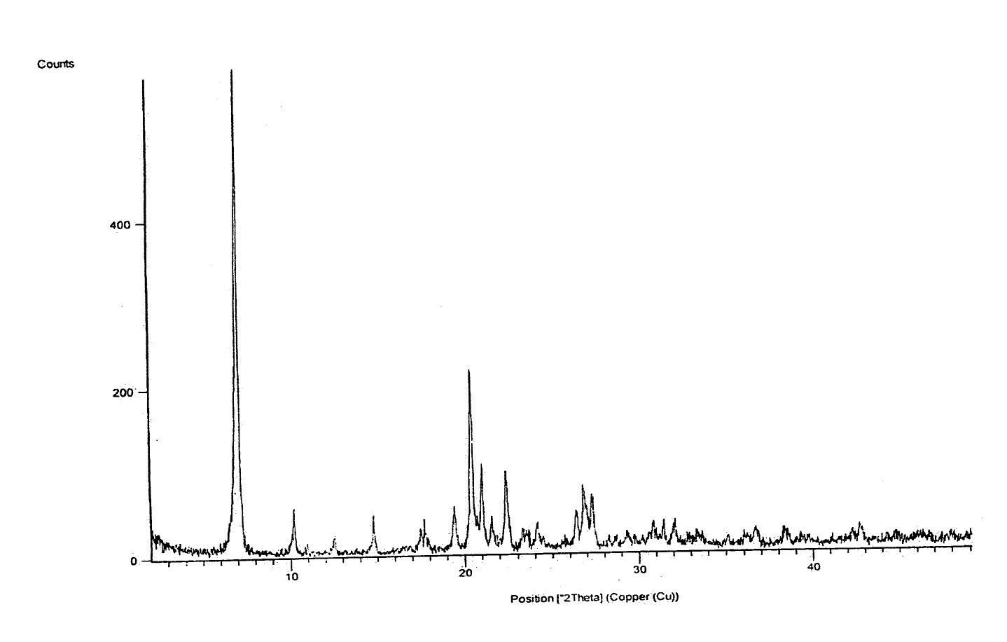

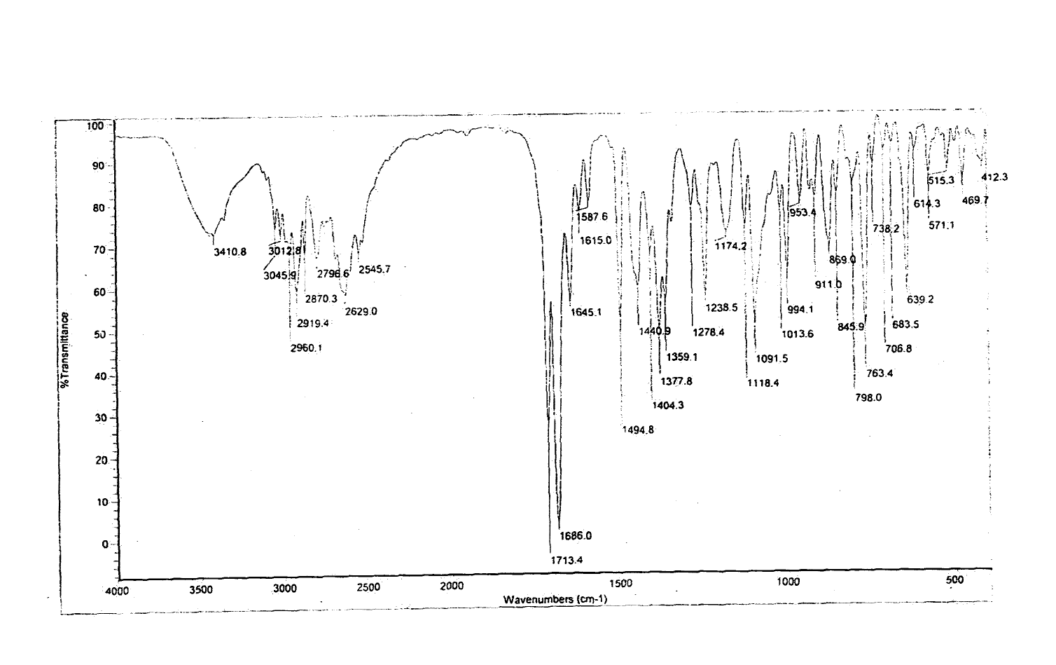

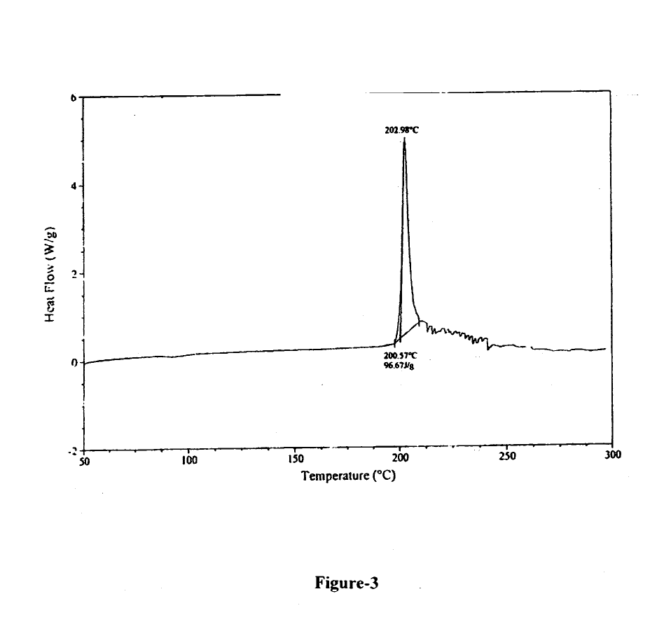

The crystalline form of the present invention is herein designated as crystalline form M. The novel crystalline form-M of the present invention is characterized by its Powder X-ray diffractogram having characteristic 2θ peaks at 7.07, 10.18, 14.81, 19.41, 20.44, 21.03, 22.37, 26.39, 26.86 and 27.32±0.2 degrees 2θ as illustrated in FIG. 1; and by its Infra-Red spectrum having peaks at 3410.8, 3045.9, 2919.4, 2629.0, 2545.7, 1713.4, 1686.0, 1494.8, 1377.8, 1174.2, 1091.5, 1013.6, 1091.5 and 798.0 cm⁻¹ as illustrated in FIG. 2 and Differential Scanning Calorimetry showing exothermic peak at 202.98° C. as illustrated in FIG. 3.

The present invention also provides a process for the preparation of crystalline form-M of compound of formula-8a, which comprises of treating the 5-(α-cyclopropylcarbonyl-2-fluorobenzyl)-2-oxo-2,4,5,6,7,7a-hexahydro thieno[3,2-c] pyridine in a suitable ketone solvent, preferably acetone with aqueous hydrobromic acid or hydrobromic acid in a suitable ester or alcohol solvent, at a suitable temperature ranges from 0 to 20° C., preferably 0-5° C. to provide the crystalline form M of 5-(α-cyclopropylcarbonyl-2-fluorobenzyl)-2-oxo-2,4,5,6,7,7a-hexahydrothieno[3,2-c] pyridine hydrobromide.

The seventh aspect of the present invention provides the use of novel salts of 5-(α-cyclopropylcarbonyl-2-fluorobenzyl)-2-oxo-2,4,5,6,7,7a-hexahydrothieno[3,2-c] pyridine of the present invention and crystalline form-M of 5-(α-cyclopropylcarbonyl-2-fluorobenzyl)-2-oxo-2,4,5,6,7,7a-hexahydrothieno[3,2-c] pyridine hydrobromide in the preparation of highly pure 5-(α-cyclopropylcarbonyl-2-fluorobenzyl)-2-oxo-2,4,5,6,7,7a-hexahydro thieno[3,2-c] pyridine as well as in the preparation of highly pure prasugrel and its pharmaceutically acceptable salts.

The eighth aspect of the present invention provides a crystalline form of 5-(α-cyclopropylcarbonyl-2-fluorobenzyl)-2-oxo-2,4,5,6,7,7a-hexahydrothieno[3,2-c] pyridine having the following structural formula

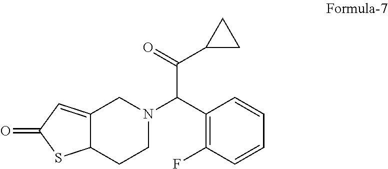

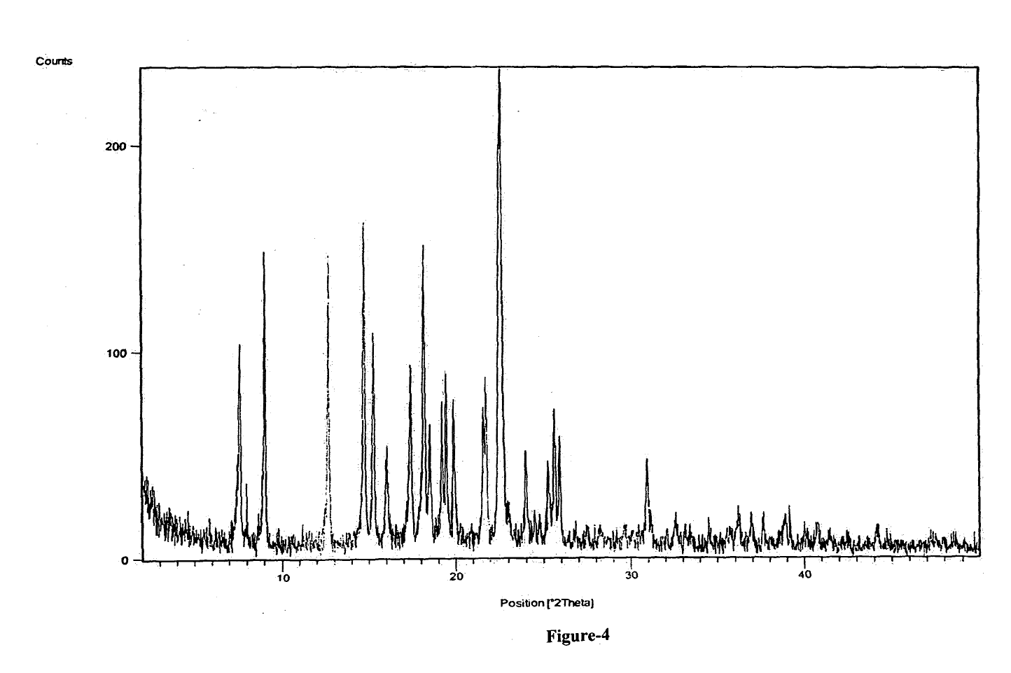

The novel crystalline form of the present invention herein designated as form-S. The crystalline form-S of the present invention is characterized by its Powder X-ray diffractogram having characteristic 2θ peaks at 7.63, 9.07, 12.73, 14.78, 15.30, 17.43, 18.20, 18.53, 19.47, 19.89, 21.70, 22.58, 24.00, 30.92±0.2 degrees 2θ and the same has been illustrated in FIG. 4.

The present invention also provides a process for the purification and crystallization of 5-(α-cyclopropylcarbonyl-2-fluorobenzyl)-2-oxo-2,4,5,6,7,7a-hexahydrothieno[3,2-c] pyridine, which comprises of treating the crude 5-(α-cyclopropylcarbonyl-2-fluorobenzyl)-2-oxo-2,4,5,6,7,7a-hexahydrothieno[3,2-c] pyridine compound of formula-7 with a suitable acid as defined above in a suitable solvent selected from alcohols, ketones, esters, hydrocarbons, polar aprotic solvents, polar solvents, chloro solvents, nitriles or mixtures thereof to provide the corresponding salt of 5-(α-cyclopropylcarbonyl-2-fluorobenzyl)-2-oxo-2,4,5,6,7,7a-hexahydro thieno[3,2-c] pyridine compound of general formula-8,

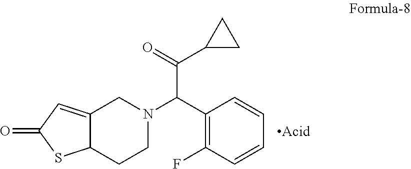

Wherein “acid” is as defined above;

Treating the thus obtained salt compound of formula-8 with a suitable inorganic or organic base, preferably inorganic base like sodium bicarbonate in a suitable solvent selected from alcohols, ketones, esters, hydrocarbons, polar aprotic solvents, polar solvents, chloro solvents, nitriles or mixtures thereof to provide pure crystalline 5-(α-cyclopropylcarbonyl-2-fluorobenzyl)-2-oxo-2,4,5,6,7,7a-hexahydro thieno[3,2-c] pyridine. The PXRD of the obtained crystalline form is shown in FIG. 4.

In the preferred embodiment of the present invention, the crystallization/purification of 5-(α-cyclopropylcarbonyl-2-fluorobenzyl)-2-oxo-2,4,5,6,7,7a-hexahydro thieno[3,2-c] pyridine process comprises of the following steps;
(a) Reacting the 5-(α-cyclopropylcarbonyl-2-fluorobenzyl)-2-oxo-2,4,5,6,7,7a-hexahydro thieno[3,2-c] pyridine with hydrobromic acid in acetone to provide the hydrobromide salt of 5-(α-cyclopropylcarbonyl-2-fluorobenzyl)-2-oxo-2,4,5,6,7,7a-hexahydro thieno[3,2-c] pyridine,
(b) treating the above obtained hydrobromide salt compound with aqueous sodium bicarbonate at 10-15° C.,
(c) stirring the reaction mixture for 30 minutes at 10-15° C.,
(d) extracting the 5-(α-cyclopropylcarbonyl-2-fluorobenzyl)-2-oxo-2,4,5,6,7,7a-hexahydro thieno[3,2-c] pyridine in to suitable solvent like methylene chloride,
(e) distilling off the solvent under reduced pressure at below 55-60° C.,
(f) adding a suitable hydrocarbon solvent, preferably cyclohexane to the obtained residue,
(g) stirring the reaction mixture for an hour at 25-30° C.,
(h) filtering and washing the solid then drying the solid to provide highly pure crystalline 5-(α-cyclopropylcarbonyl-2-fluorobenzyl)-2-oxo-2,4,5,6,7,7a-hexahydro thieno[3,2-c] pyridine.

The ninth aspect of the present invention provides a novel crystalline form of prasugrel free base compound of formula-1.

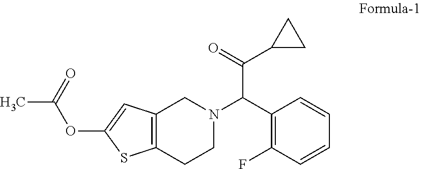

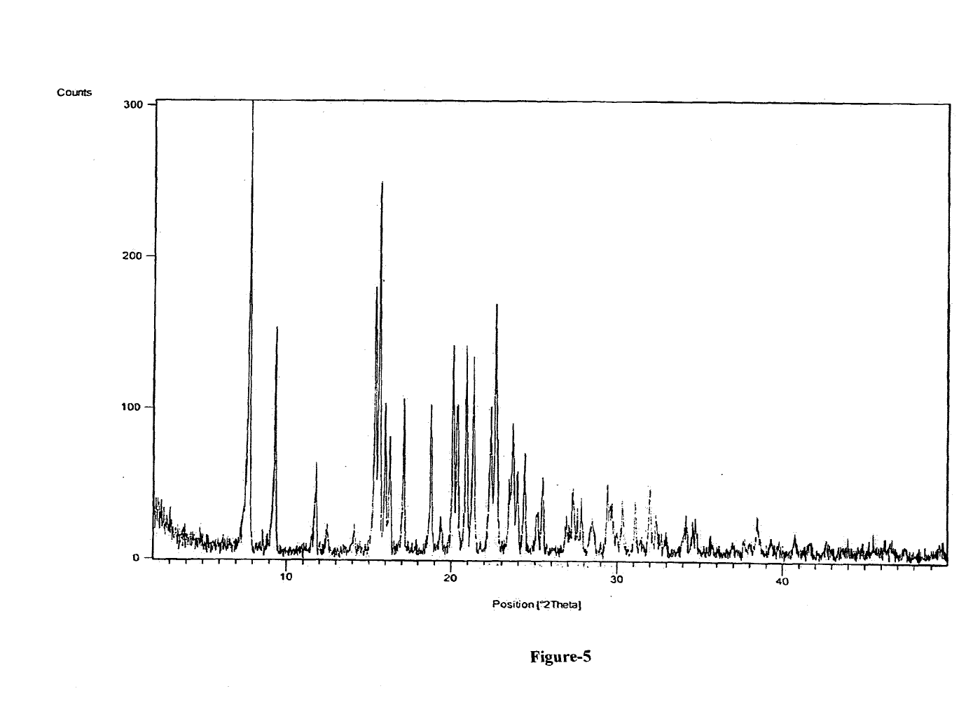

The novel crystalline form of the present invention herein is designated as form-N. The crystalline form-N of the present invention is characterized by its Powder X-ray diffractogram having characteristic 2θ peaks at 7.80, 9.35, 11.79, 15.38, 15.64, 15.98, 16.28, 17.14, 18.78, 20.10, 20.36, 20.91, 21.35, 22.35, 22.63, 23.59, 24.39, 25.51, 29.43, 31.08, 31.99±0.2 degrees 2θ and the same has been illustrated in FIG. 5.

The tenth aspect of the present invention provides an improved process for the preparation of prasugrel compound of formula-1

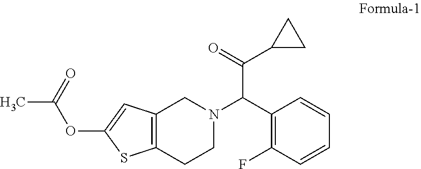

and its pharmaceutically acceptable salts, which comprises of acetylating the 5-(α-cyclopropylcarbonyl-2-fluorobenzyl)-2-oxo-2,4,5,6,7,7a-hexahydrothieno[3,2-c] pyridine with a suitable acetylating agent like acetic anhydride, in presence of a suitable organic base selected from triethyl amine, tributyl amine, pyridine, 4-dimethylaminopyridine, N-methylmorpholine and diisopropylethyl amine and the like, preferably triethylamine in a suitable hydrocarbon solvent, chloro solvent preferably toluene, followed by crystallization from a suitable alcoholic solvent to provide prasugrel compound of formula-1. Optionally converting the obtained prasugrel into its pharmaceutically acceptable salts by the process known in the art.

In a preferred embodiment of the present aspect of the invention, the process for the preparation of prasugrel compound of formula-1 comprises of reacting the 5-(α-cyclopropylcarbonyl-2-fluorobenzyl)-2-oxo-2,4,5,6,7,7a-hexahydrothieno[3,2-c] pyridine compound of formula-7 with acetic anhydride in presence of triethyl amine in toluene at a temperature ranges from 0-30° C., preferably 25-30° C. followed isolating the prasugrel compound of formula-1 from methanol.

The eleventh aspect of the invention provides an improved process for the preparation of prasugrel and its pharmaceutically acceptable salts, which comprises of;
a) protecting the amino functional group of 4,5,6,7-tetrahydrothieno[3,2-c] pyridine hydrochloride compound of formula-2a,

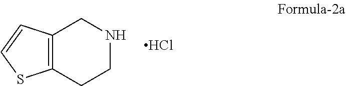

with triphenyl methyl chloride in presence of a suitable base like triethyl amine in a suitable solvent like methylene chloride, followed by crystallization from a suitable alcoholic solvents selected from methanol, ethanol, propanol, isopropyl alcohol and butanol or mixtures thereof, preferably isopropyl alcohol to provide a compound of formula-10,

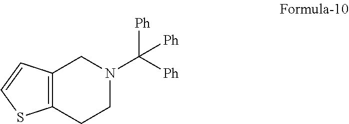

b) reacting the compound of formula-10 with n-butyl lithium in tetrahydrofuran at 0-5° C. under nitrogen atmosphere, followed by treating the reaction mixture with tri-n-butyl borate and then treating with hydrogen peroxide, followed by crystallization from a suitable alcoholic solvents like methanol, ethanol, propanol, isopropyl alcohol and butanol or mixtures thereof, preferably isopropyl alcohol to provide a compound of formula-11,

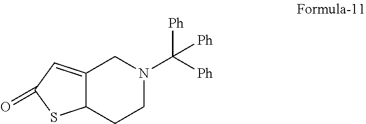

c) treating the compound of formula-11 with a suitable acid like hydrochloric acid or p-toluene sulfonic acid in a suitable solvent like acetone, followed by crystallization from acetone to provide the corresponding acid addition salt of 5,6,7,7a-tetrahydro-4H-thieno[3,2-c]pyridine-2-one compound of general formula-12,

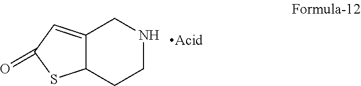

12a) Acid=PTSA and 12b) Acid=HCl
d) Optionally purifying the compound of formula-12 by slurrying with a suitable solvent or with a mixture of solvents
e) reacting the 5,6,7,7a-tetrahydro-4H-thieno[3,2-c]pyridine-2-one acid addition salt compound of formula-12 or its free base with α-cyclopropylcarbonyl-2-fluorobenzyl bromide compound of formula-3,

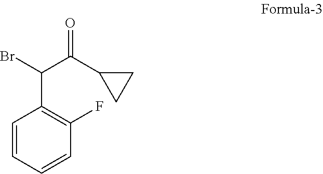

in the presence of potassium carbonate in acetonitrile, followed by crystallization from a mixture of ethyl acetate and cyclohexane to provide compound of formula-7,

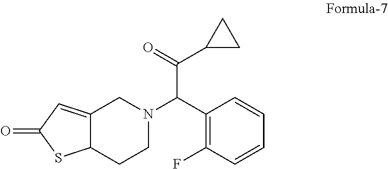

f) acetylating the compound of formula-7 with a suitable acetylating agent like acetic anhydride in a suitable solvent selected from hydrocarbon solvents like toluene, xylene, heptane, cyclohexane and hexane, preferably toluene in presence of a suitable organic base, preferably triethylamine, followed by crystallization/isolation from a suitable alcoholic solvent, preferably methanol to provide the compound of formula-1,

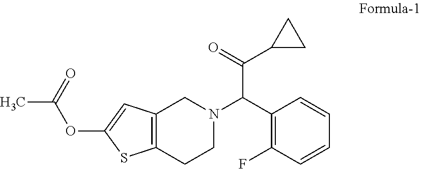

g) optionally converting the prasugrel into its hydrochloric acid salt by treating it with HCl gas or HCl gas dissolved in organic solvent like ethyl acetate, isopropyl acetate preferably ethyl acetate hydrochloride provides prasugrel hydrochloride salt compound of formula-1a.

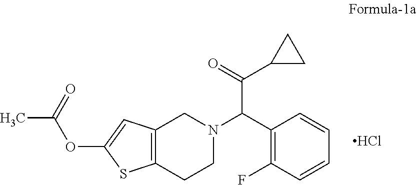

In a preferred embodiment the present invention also provides a one-pot process for the preparation of prasugrel compound of formula-1, which comprises of
a) Reacting the 5,6,7,7a-tetrahydro-4H-thieno[3,2-c]pyridine-2-one acid addition salt compound of formula-12

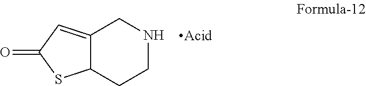

12a) ACID=PTSA and 12b) ACID=HCl
   or its free base with a-cyclopropylcarbonyl-2-fluorobenzyl bromide compound of formula-3,

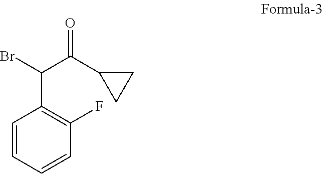

in presence of suitable organic or inorganic bases, preferably inorganic base in a suitable solvent selected from nitrile solvent, ketone solvent, ester solvent, polar aprotic solvent or mixtures thereof, preferably nitrile solvent like acetonitrile,
b) filtering the unwanted solid and washing with a suitable solvent,
c) treating the filtrate containing 5-(α-cyclopropylcarbonyl-2-fluorobenzyl)-2-oxo-2,4,5,6,7,7a-hexahydrothieno[3,2-c] pyridine compound of formula-7 with acetic anhydride in presence of a suitable organic base, preferably triethylamine,
d) extracting the reaction mixture into suitable hydrocarbon solvent preferably toluene,
e) distilling off the solvent completely under reduced pressure,
f) crystallizing the obtained residue from a suitable alcoholic solvent to provide the prasugrel compound of formula-1

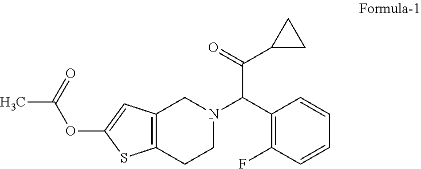

The above obtained prasugrel optionally is converted into its hydrochloric acid salt by treating it with HCl gas or HCl gas dissolved in organic solvent like ethyl acetate, isopropyl acetate preferably ethyl acetate hydrochloride to provide prasugrel hydrochloride salt compound of formula-1a.

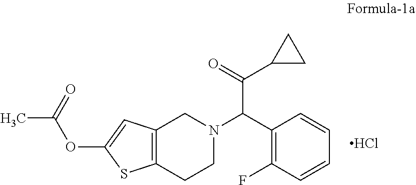

Prasugrel and its pharmaceutically acceptable salts of the present invention can be micronized or milled to get the desired particle size. The present invention is schematically represented as follows.

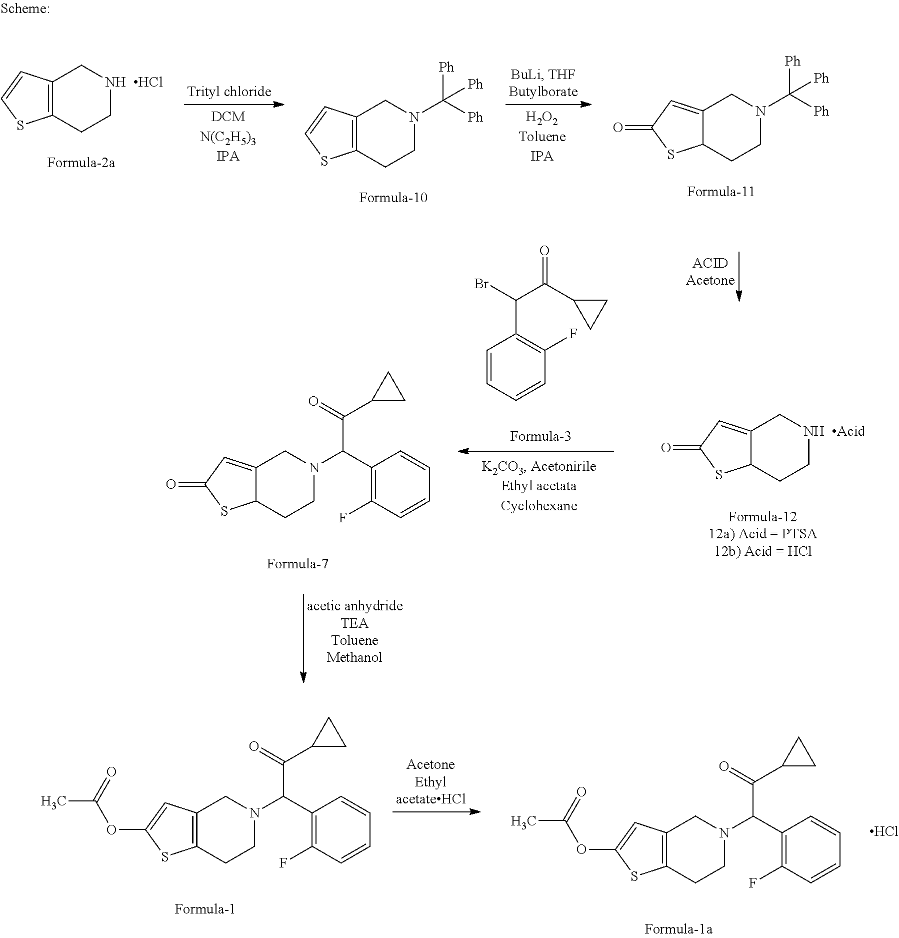

The twelfth aspect of the present invention provides a novel process for the preparation of 1-cyclopropyl-2-(2-fluorophenyl)ethanone compound of formula-16,

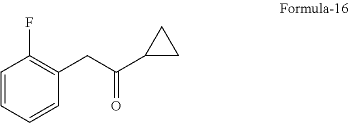

which comprise the following steps;
a) Reacting the 2-(2-fluorophenyl)acetic acid compound of formula-13

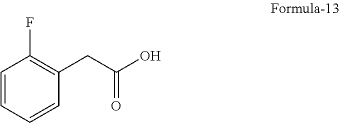

with N,O-dialkylhydroxylamine or its salts compound of general formula-14,

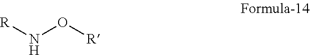

Wherein R and R′ each independently represents C₁₋₆ alkyl group, having a straight chain or branched chain,
in the presence of a base in a suitable solvent provides 2-(2-fluorophenyl)-N-alkoxy-N-alkylacetamide compound of general formula-15,

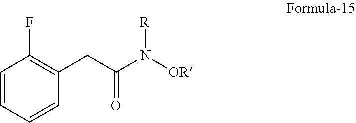

Wherein R and R′ each independently represents C₁₋₆ alkyl group, having a straight chain or branched chain;
b) reacting the 2-(2-fluorophenyl)-N-alkoxy-N-alkylacetamide compound of general formula-15 with cyclopropyl magnesium bromide in a suitable solvent to provide 1-cyclopropyl-2-(2-fluorophenyl)ethanone compound of formula-16.

Wherein in step a) the reaction between 2-(2-fluorophenyl)acetic acid compound of formula-13 with N,O-dialkylhydroxylamine or its salts compound of general formula-14 is carried out with suitable reagent selected from N,N′-Dicyclohexyl carbodiimide (DCC) in presence of hydroxybenzotriazole (HOBT), or N,N′-Dicyclohexyl carbodiimide (DCC) in presence of 4-Dimethylaminopyridine (DMAP) or thionyl chloride, phosphorous pentachloride; preferably DCC in presence of HOBT.; the suitable base is selected from alkali metal hydroxides, alkali metal carbonates, alkali metal bicarbonates, alkali metal alkoxides or the organic bases like triethyl amine, diisopropyl ethylamine preferably tri ethyl amine; and the suitable solvent is selected from alcohol solvents, ester solvents, hydrocarbon solvents, ketone solvents, nitrile solvents, chloro solvents, polar aprotic solvents, polar solvents or mixtures thereof; preferably methylene chloride;

Wherein in step b) the suitable solvent is selected from ester solvents, ether solvents, hydrocarbon solvents, polar aprotic solvents, ketone solvents, alcoholic solvents, chloro solvents, polar solvents and mixtures thereof.

In a preferred embodiment, the process for the preparation of compound of formula-16 comprises of the following steps;
d) Reacting the 2-(2-fluorophenyl)acetic acid compound of formula-13

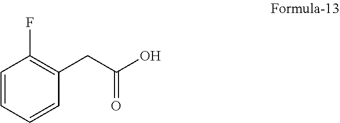

with N,O-dimethylhydroxylamine or its salts compound of formula-14a,

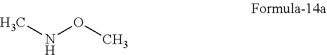

in the presence of dicyclohexylcarbodiimide (DCC) & 1-hydroxybenzotriazole (HOBt) and in the presence of triethyl amine in methylene chloride provides 2-(2-fluorophenyl)-N-methoxy-N-methylacetamide compound of formula-15a,

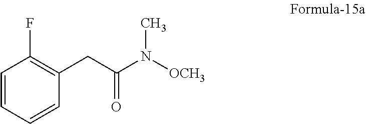

e) reacting the 2-(2-fluorophenyl)-N-methoxy-N-methylacetamide compound of formula-15a with cyclopropyl magnesium bromide in tetrahydrofuran to provide 1-cyclopropyl-2-(2-fluorophenyl)ethanone compound of formula-16.

Further the present invention also provides a novel 2-(2-fluorophenyl)-N-alkoxy-N-alkylacetamide compound represented by the following general structural formula-15

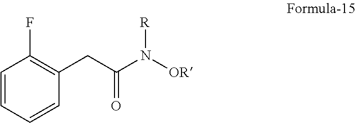

Wherein R and R′ each independently represents C₁₋₆ alkyl group, having a straight chain or branched chain.
The novel compound of formula-15 of the present invention is used to prepare the compound of formula-16 and prasugrel or its pharmaceutically acceptable salts.

In a preferred embodiment, the present invention provides the 2-(2-fluorophenyl)-N-methoxy-N-methylacetamide compound of formula-15a.

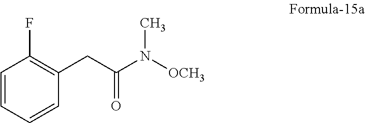

The 1-cyclopropyl-2-(2-fluorophenyl)ethanone compound of formula-16 prepared as per the present invention is obtained in a good yield and purity and free of 1-methyl-2-(2-fluorophenyl)ethanone impurity. Hence the usage of compound of formula-16 obtained in the present invention in the preparation of prasugrel avoids the formation of corresponding impurities (especially methyl keto impurity).

The thirteenth aspect of the present invention provides a process for the preparation of highly pure prasugrel compound of formula-1, which comprises of the following steps;
a) Reacting the 2-(2-fluorophenyl)acetic acid compound of formula-13

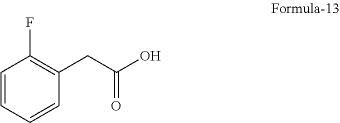

with N,O-dialkylhydroxylamine or its salts compound of general formula-14,

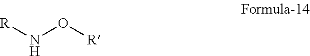

Wherein R and R′ each independently represents C₁₋₆ alkyl group, having a straight chain or branched chain,
in the presence of a base in a suitable solvent provides 2-(2-fluorophenyl)-N-alkoxy-N-alkylacetamide compound of general formula-15,

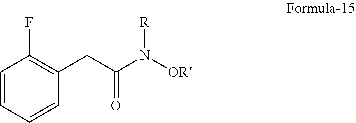

Wherein R and R′ each independently represents C₁₋₆ alkyl group, having a straight chain or branched chain;
b) reacting the 2-(2-fluorophenyl)-N-alkoxy-N-alkylacetamide compound of general formula-15 with cyclopropyl magnesium bromide in a suitable solvent to provide 1-cyclopropyl-2-(2-fluorophenyl)ethanone compound of formula-16,

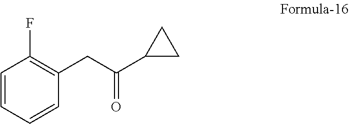

c) reacting the compound of formula-16 with a suitable brominating agent in presence or absence of base in a suitable solvent to provide 2-bromo-1-cyclopropyl-2-(2-fluorophenyl)ethanone compound of formula-3,

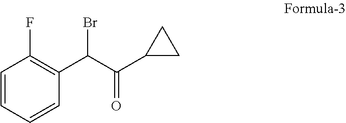

d) reacting the compound of formula-3 with 5,6,7,7a-tetrahydrothieno[3,2-c]pyridin-2(4H)-one compound of formula-17,

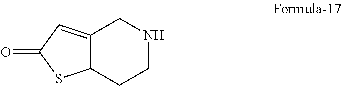

in the presence of suitable base and in a suitable solvent provides 5-(2-cyclopropyl-1-(2-fluorophenyl)-2-oxoethyl)-5,6,7,7a-tetrahydrothieno[3,2-c]pyridin-2(4H)-one compound of formula-7,

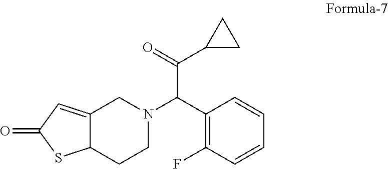

e) acetylating the compound of formula-7 with a suitable acetylating agent in presence of suitable base in a suitable solvent provides 5-(2-cyclopropyl-1-(2-fluorophenyl)-2-oxoethyl)-4,5,6,7-tetrahydrothieno[3,2-c]pyridin-2-yl acetate compound of formula-1,

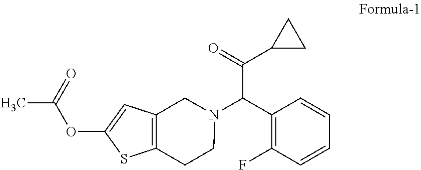

f) optionally purifying the compound of formula-1 using suitable solvents and converting it into its pharmaceutically acceptable salts.

The fourteenth aspect of the present invention provides a process for the purification of 1-cyclopropyl-2-(2-fluorophenyl) ethanone compound of formula-16, which comprises subjecting the crude 1-cyclopropyl-2-(2-fluorophenyl) ethanone to distillation under reduced pressure (high vacuum distillation (HVD) to obtain pure 1-cyclopropyl-2-(2-fluorophenyl) ethanone. The purification is carried out by fractional distillation and the pure compound fractions of formula-16 obtained at a vapour temperature of 80-90° C.

The compound of formula-16 prepared as per the prior art process containing 1-methyl-2-(2-fluorophenyl)ethanone having the following structural formula-18

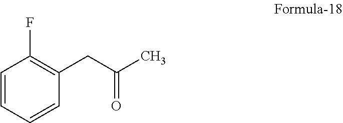

as an impurity at the level of 1 to 5%. The same has been discussed when subjecting the compound of formula-16 into the high vacuum distillation and the pure fraction of compound of formula-16 collected at a vapor temperature of 80-90° C. and the impurity fraction collected at different temperature. According to the present invention the impurity level brought down to 0.5 to 0.1% even to level of non detection from the level of 5% by GC. When using this compound of formula-16 (having least amount of impurity), in the preparation of prasugrel provides highly pure prasugrel containing the corresponding derivative (i.e. 2-acetoxy-5-(α-methyl carbonyl-2-fluorobenzyl)-4,5,6,7-tetrahydro thieno[3,2-c] pyridine) to the lowest level i.e., less than 0.15%, preferably less than 0.05% and more preferably less than 0.01% by HPLC.

The fifteenth aspect of the present invention provides 1-cyclopropyl-2-(2-fluorophenyl)ethanone compound of formula-16 containing less than 4.0% of 1-methyl-2-(2-fluorophenyl)ethanone by GC; preferably less than 1.0% by GC and more preferably 0.1% by GC.

Further, the sixteenth aspect of the present invention provides 5-(2-cyclopropyl-1-(2-fluorophenyl)-2-oxoethyl)-4,5,6,7-tetrahydrothieno[3,2-c]pyridin-2-ylacetate compound of formula-1 and its pharmaceutically acceptable salts containing less than 3.0% of 2-acetoxy-5-(α-methyl carbonyl-2-fluorobenzyl)-4,5,6,7-tetrahydrothieno[3,2-c] pyridine (methyl keto impurity) by HPLC, preferably less than 1.0% by HPLC and more preferably less than 0.1% by HPLC.

The seventeenth aspect of the present invention provides a process for the purification of 5-(2-cyclopropyl-1-(2-fluorophenyl)-2-oxoethyl)-4,5,6,7-tetrahydrothieno[3,2-c] pyridin-2-yl acetate compound of formula-1, which comprises of crystallizing the compound of formula-1 using nitrile or alcohol solvent alone or their mixture. Preferably using a mixture of acetonitrile and isopropyl alcohol solvents. The prasugrel prepared by this process having purity greater than 99.15% by HPLC and preferably greater than 99.50% by HPLC.

The present inventors observed the enhancement of des-acetyl impurity in normal packing conditions of Prasugrel hydrochloride.

There is need in the art to provide an improved packing conditions to control the said des-acetyl impurity.

Accordingly the present inventors developed an improved packing conditions which controls the des-acetyl impurity.

An improved packing of Prasugrel hydrochloride to control des-acetyl impurity and other impurities comprises of the following steps
a. Packing the prasugrel hydrochloride in clear low-density polyethylene bag and sealing the bag with vacuum sealer,
b. Placing the above bag in black color low-density polyethylene bag and seal the bag with vacuum sealer,
c. Placing the above bag in triple laminated bag and sealing the bag with vacuum sealer,
d. Placing the above bag in HDPE container and sealing the container,
e. Storing the container at control room temperature.

The present invention is schematically represented as follows

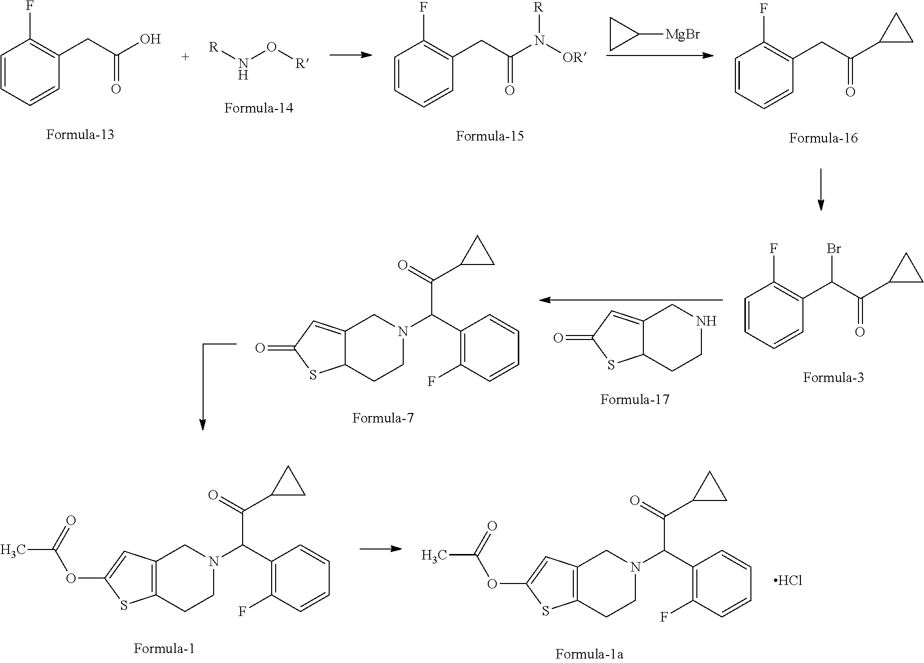

Related substances of the 1-cyclopropyl-2-(2-fluorophenyl)ethanone is measured by Gas Chromatography using the following conditions. Apparatus: A gas chromatographic system is to be equipped with FID; Column: DB-1 column or equivalent; Length: 30 mts; ID: 0.53 mm; Film thickness: 3.0 μm; Injector temperature: 220° C.; Split ratio: 1:50; Detector temperature: 260° C(FID); Carrier gas: Helium; Carrier gas pressure: 3.0 PSI; Injection volume: 0.1 μl.

Related substances of the prasugrel and its pharmaceutically acceptable salts were measured by High Performance Liquid Chromatography (HPLC) using the following conditions.

Apparatus: A liquid chromatograph is equipped with variable wavelength UV-Detector; Column: ZORBAX SB-Phenyl, 250×4.6 mm ID, 5 μm or equivalent; Flow rate: 1.20 ml/min, wave length: 235 nm, Temperature: 30° C.; load: 30 ml; Run time: 60 minutes; Diluent: Mobile phase-B, Elution: Gradient

PXRD analysis of prasugrel, its intermediates and their salts were carried out using SIEMENS/D-5000 X-Ray diffractometer using Cu, Ka radiation of wavelength 1.54 A° and continuous scan speed of 0.045°/min. FI-IR spectrum of prasugrel and its intermediate were recorded on Thermo model Nicolet-380 as KBr pellet. The thermal analysis of prasugrel and intermediates were carried out on Waters DSC Q-10 model differential scanning calorimeter.

The process described in the present invention was demonstrated in examples illustrated below. These examples are provided as illustration only and therefore should not be construed as limitation of the scope of the invention.

### EXAMPLES

### Example 1

### Preparation of 5-(α-cyclopropylcarbonyl-2-fluorobenzyl)-4,5,6,7,-tetrahydrothieno[3,2-c] pyridine Compound of Formula-4

2-fluoro-α-cyclopropyl carbonyl benzyl bromide (6.1 grams) was added to a mixture of 4,5,6,7-tetrahydrothieno[3,2-c] pyridine hydrochloride (5.0 grams) and potassium carbonate (6.0 grams) in acetonitrile (50 ml) at temperature 25 to 35° C. and stirred for 5 hours. The reaction mixture was filtered and the filtrate was distilled off completely. The obtained residue was purified using cyclohexane and ethyl acetate to provide the title compound.

Yield: 8.0 grams

### Example 2

### Preparation of 5-(α-cyclopropylcarbonyl-2-fluorobenzyl)-4,5,6,7,-tetrahydrothieno[3,2-c] pyridine Compound of Formula-4

2-fluoro-α-cyclopropyl carbonyl benzyl bromide (6.1 grams) was added to a mixture of 4,5,6,7-tetrahydrothieno[3,2-c] pyridine hydrochloride (5.0 grams) and sodium carbonate (5.0 grams) in acetonitrile (50 ml) at temperature 25 to 35° C. and stirred for 5 hours. The reaction mixture was filtered and the filtrate was distilled off completely. The obtained residue was purified using cyclohexane and ethyl acetate to provide the title compound.

Yield: 7.9 grams.

### Example 3

### Preparation of 5-(α-cyclopropylcarbonyl-2-fluorobenzyl)-4,5,6,7,-tetrahydrothieno[3,2-c] pyridine hydro bromide

To 5-(α-cyclopropylcarbonyl-2-fluorobenzyl)-4,5,6,7,-tetrahydrothieno[3,2-c] pyridine (25 grams) added acetone (50 ml) and stirred for 15 minutes. Cool the reaction mixture to 0-5° C. Added aqueous hydro bromide (13 ml) slowly to the reaction mixture. Raised the temperature to 25-30° C. and stirred for 3 hours. Cooled the reaction mass to 0-5° C. and stirred for 2 hours at same temperature. Filtered the reaction mixture and washed with acetone. Dried the material to get the title compound.

Yield: 29 grams

### Example 4

### Preparation of 5-(α-cyclopropylcarbonyl-2-fluorobenzyl)-2-oxo-2,4,5,6,7,7a-hexahydrothieno[3,2-c]pyridine Compound of Formula-7

To a solution of 5-(α-cyclopropylcarbonyl-2-fluorobenzyl)-4,5,6,7,-tetrahydrothieno[3,2-c] pyridine(5.0 g) in tetrahydrofuran(100 ml) added a solution of lithium diisopropylamide (1.86 g) in tetrahydrofuran dropwise at −78° C. Stirred the reaction mixture for 1 hr at −78° C. and added tetramethylurea and n-butyl lithium (22 ml) at same temperature. Stirred the reaction mixture for 1 hr and tri methyl borate (4 ml) was added to it at −60° C. Slowly warmed the reaction mixture to 0° C. and added (2.1 ml) of hydrogen peroxide to it. Stirred the reaction mixture for further 1 hr at 0° C. 25 ml of water was added to the reaction mixture and extracted with 60 ml of methyl tertiary butyl ether. Separated the both aqueous and organic layers. Neutralized the P^{H} using aqueous hydrochloric acid and organic layer was washed with brine solution. Distilled off the solvent completely from organic layer to get the title compound.

Yield: 2.7 grams.

### Example 5

### Preparation of 5-(α-cyclopropylcarbonyl-2-fluorobenzyl)-2-oxo-2,4,5,6,7,7a-hexahydrothieno[3,2-c]pyridine Compound of Formula-7

To a solution of 5-(α-cyclopropylcarbonyl-2-fluorobenzyl)-4,5,6,7,-tetrahydrothieno[3,2-c] pyridine(5.0 g) in tetrahydrofuran(100 ml) added a solution of lithium diisopropylamide (1.86 g) in tetrahydrofuran dropwise at −78° C. Stirred the reaction mixture for 1 hr at −78° C. and added tetramethylurea and n-butyl lithium (22 ml) at same temperature. Stirred the reaction mixture for 1 hr and tri n-butylborate (9.5 ml) was added to it at −60° C. Slowly warmed the reaction mixture to 0° C. and added (2.1 ml) of hydrogen peroxide to it. Stirred the reaction mixture for further 1 hr at 0° C. 25 ml of water was added to the reaction mixture and extracted with 60 ml of methyl tertiary butyl ether. Separated the both aqueous and organic layers. Neutralized the P^{H} using aqueous hydrochloric acid and organic layer was washed with brine solution. Distilled off the solvent completely from organic layer to get the title compound.

Yield: 3.0 grams

### Example 6

### Preparation of 5-(α-cyclopropylcarbonyl-2-fluorobenzyl)-2-oxo-2,4,5,6,7,7a-hexahydrothieno[3,2-c]pyridine Compound of Formula-7

To a solution of 5-(α-cyclopropylcarbonyl-2-fluorobenzyl)-4,5,6,7,-tetrahydrothieno[3,2-c] pyridine(5.0 g) in tetrahydrofuran(100 ml) added a solution of lithium diisopropylamide (1.86 g) in tetrahydrofuran dropwise at −78° C. Stirred the reaction mixture for 1 hr at −78° C. and added 1,3-Dimethyl-3,4,5,6-tetrahydro-2(1H)-pyrimidinone(4.2 ml) and n-butyl lithium (22 ml) at same temperature. Stirred the reaction mixture for 1 hr and tri n-butylborate (9.5 ml) was added to it at −60° C. Slowly warmed the reaction mixture to 0° C. and added (2.1 ml) of hydrogen peroxide to it. Stirred the reaction mixture for further 1 hr at 0° C. 25 ml of water was added to the reaction mixture and extracted with 60 ml of ethyl acetate. Separated the both aqueous and organic layers. Neutralized the P^{H} using aqueous hydrochloric acid and organic layer was washed with brine solution. Distilled off the solvent completely from organic layer to get the title compound.

Yield: 2.9 grams.

### Example 7

### Preparation of 5-(α-cyclopropylcarbonyl-2-fluorobenzyl)-2-oxo-2,4,5,6,7,7a-hexahydrothieno[3,2-c]pyridine Compound of Formula-7

To a solution of 5-(α-cyclopropylcarbonyl-2-fluorobenzyl)-4,5,6,7,-tetrahydrothieno[3,2-c] pyridine(5.0 g) in tetrahydrofuran(100 ml) added a solution of lithium diisopropylamide (1.86 g) in tetrahydrofuran dropwise at −78° C. Stirred the reaction mixture for 1 hr at −78° C. and added n-butyl lithium (40 ml) at same temperature. Stirred the reaction mixture for 1 hr and tri n-butylborate (9.5 ml) was added to it at −60° C. Slowly warmed the reaction mixture to 0° C. and added (2.1 ml) of hydrogen peroxide to it. Stirred the reaction mixture for further 1 hr at 0° C. 25 ml of water was added to the reaction mixture and extracted with 60 ml of methyl tertiary butyl ether. Separated the both aqueous and organic layers. Neutralized the P^{H} using aqueous hydrochloric acid and organic layer was washed with brine solution. Distilled off the solvent completely from organic layer to get the title compound. Yield: 3.2 grams.

### Example 8

### Preparation of 5-(α-cyclopropylcarbonyl-2-fluorobenzyl)-2-oxo-2,4,5,6,7,7a-hexahydrothieno[3,2-c]pyridine Compound of Formula-7

To a solution of 5-(α-cyclopropylcarbonyl-2-fluorobenzyl)-4,5,6,7,-tetrahydrothieno[3,2-c] pyridine(5.0 g) in tetrahydrofuran(100 ml) added a solution of lithium diisopropylamide (1.86 g) in tetrahydrofuran dropwise at −78° C. Stirred the reaction mixture for 1 hr at −78° C. and added lithium hexamethyldisilazide (80 ml) at same temperature. Stirred the reaction mixture for 1 hr and tri n-butylborate (9.5 ml) was added to it at −60° C. Slowly warmed the reaction mixture to 0° C. and added (2.1 ml) of hydrogen peroxide to it. Stirred the reaction mixture for further 1 hr at 0° C. 25 ml of water was added to the reaction mixture and extracted with 60 ml of methyl tertiary butyl ether. Separated the both aqueous and organic layers. Neutralized the P^{H} using aqueous hydrochloric acid and organic layer was washed with brine solution. Distilled off the solvent completely from organic layer to get the title compound.

Yield: 2.6 grams.

### Example 9

### Preparation of 5-(α-cyclopropylcarbonyl-2-fluorobenzyl)-2-oxo-2,4,5,6,7,7a-hexahydrothieno[3,2-c]pyridine Compound of Formula-7

To a solution of 5-(α-cyclopropylcarbonyl-2-fluorobenzyl)-4,5,6,7,-tetrahydro thieno[3,2-c] pyridine(5.0 g) in tetrahydrofuran(100 ml) added a solution of lithium diisopropylamide (2.54 g) in tetrahydrofuran dropwise at −78° C. Stirred the reaction mixture for 1 hr at −78° C. and added tetramethylurea (4.5 ml),and n-butyl lithium (40 ml) at same temperature. Stirred the reaction mixture for 1 hr and tri n-butylborate (9.5 ml) was added to it at −60° C. Slowly warmed the reaction mixture to 0° C. and added (2.1 ml) of hydrogen peroxide to it. Stirred the reaction mixture for further 1 hr at 0° C. 25 ml of water was added to the reaction mixture and extracted with 60 ml of methyl tertiary butyl ether. Separated the both aqueous and organic layers. Neutralized the P^{H} using aqueous hydrochloric acid and organic layer was washed with brine solution. Distilled off the solvent completely from organic layer to get the title compound.

Yield: 3.4 grams.

### Example 10

### Preparation of 5-(α-cyclopropylcarbonyl-2-fluorobenzyl)-2-oxo-2,4,5,6,7,7a-hexahydrothieno[3,2-c]pyridine Compound of Formula-7

To a solution of 5-(α-cyclopropylcarbonyl-2-fluorobenzyl)-4,5,6,7,-tetrahydrothieno[3,2-c] pyridine(5.0 g) in tetrahydrofuran(100 ml) added a solution of n-butyl lithium (30 ml) dropwise at −78° C. Stirred the reaction mixture for 1 hr at −78° C. and added tetramethylurea (4.5 ml), n-butyl lithium (30 ml) at same temperature. Stirred the reaction mixture for 1 hr and tri n-butylborate (9.5 ml) was added to it at −60° C. Slowly warmed the reaction mixture to 0° C. and added (2.1 ml) of hydrogen peroxide to it. Stirred the reaction mixture for further 1 hr at 0° C. 25 ml of water was added to the reaction mixture and extracted with 60 ml of methyl tertiary butyl ether. Separated the both aqueous and organic layers. Neutralized the P^{H} using aqueous hydrochloric acid and organic layer was washed with brine solution. Distilled off the solvent completely from organic layer to get the title compound. Yield: 1.9 grams.

### Example 11

### Preparation of 2-Acetoxy-5-(α-cyclopropylcarbonyl-2-fluorobenzyl)-4,5,6,7-tetrahydrothieno[3,2-c] pyridine

To a solution of 5-(α-cyclopropylcarbonyl-2-fluorobenzyl)-2-oxo-2,4,5,6,7,7a-hexahydrothieno[3,2-c]pyridine (0.5 grams) dissolved in a mixture of acetonitrile (10 ml) and acetic anhydride(0.3 ml), added diisopropylethyl amine (0.55 ml). The mixture was then stirred for 20 minutes at 10-15° C. temperature, after which it was stirred for further 3 hours at 25-30° C. temperature. Ethyl acetate (20 ml) was added to the mixture, which was then washed four times, each time with 10 ml of a saturated aqueous solution of sodium chloride. The organic layer was separated and dried over anhydrous sodium sulfate, and the solvent was removed by evaporation under reduced pressure to give yellow oil. This oil was crystallized from diisopropyl ether to get the title compound as white crystals.

Yield: 0.31 grams; Melting point: 120-123° C.

### Example 12

### Preparation of 5-trityl-4,5,6,7-tetrahydrothieno [3,2-c]pyridine Compound of Formula-10

Triethylamine (181 ml) and trityl chloride (151 grams) was added a mixture of 4,5,6,7-tetrahydrothieno[3,2-c]pyridine hydrochloride (100 grams) in dichloromethane (220 ml) at 0-5° C. and stirred for 9 hours. The reaction mixture was quenched with water (300 ml), stirred at 25-30° C. then the aqueous and organic layers were separated. The solvent from the organic layer was distilled off completely under reduced pressure and isopropyl alcohol (250 ml) was added to the obtained residue and stirred for 45 minutes at reflux temperature. The reaction mixture was cooled to 25-30° C. and stirred for an hour. The solid was filtered, washed with isopropyl alcohol and dried to get the title compound.

Yield: 168 grams; M.R:160-165° C.

### Example 13

### Preparation of 5-trityl-5,6,7,7a-tetrahydrothieno[3,2-c]pyridin-2-(4H)-one Compound of Formula-11

n-butyl lithium (129 ml, 1.6 M) was added to a solution of 5-triyl-4,5,6,7-tetrahydrothieno[3,2-c] pyridine (50 grams) in tetrahydrofuran (350 ml) at 0-5° C. under nitrogen atmosphere and stirred for 1 hour at 10-15° C. The reaction mixture was cooled to 0-5° C. and tri-n-butyl borate (75 grams) in tetrahydrofuran (50 ml) was added, stirred for 1 hour at 10-15° C. Aqueous hydrogen peroxide (45 ml, 30(v/v)) was added to the reaction mixture at 0-5° C. and then stirred for 2 hours at 25-30° C. The reaction mixture was quenched with water and then extracted into toluene. The organic layer was washed with sodium sulphite solution and the solvent from it was distilled off under reduced pressure. Isopropyl alcohol (150 ml) was added to the obtained residue and stirred for 45 minutes at reflux temperature. The reaction mixture was cooled to 25-30° C. and stirred for 1 hour. The solid obtained was filtered, washed with isopropyl alcohol and dried to get the title compound.

Yield: 45 grams

### Example 14

### Preparation of 5,6,7,7a-tetrahydro-4H-thieno [3,2-c]-pyridin-2-one-p-toluenesulfonate Compound of Formula-12a

A mixture of 5-trityl-5,6,7,7a-tetrahydrothieno [3,2-c]pyridin-2(4H)-one (100 grams), acetone (1000 ml) and p-toluenesulfonic acid (48 grams) was stirred for 25 minutes at 25-30° C. The solid obtained was filtered and washed with acetone. Acetone (200 ml) was added to the wet solid and stirred for 30 min. at reflux temperature. The reaction mixture was cooled to 25-30° C. and stirred for 1 hour. The solid formed was filtered, washed with acetone and dried to get the title compound.

Yield: 78 grams; M.R:182-187° C.

### Example 15

### Preparation of 5,6,7,7a-tetrahydro-4H-thieno [3,2-c]-pyridin-2-one hydrochloride Compound of Formula-12b:

A mixture of 5-trityl-5,6,7,7a-tetrahydrothieno [3,2-c]pyridin-2(4H)-one (100 grams), acetone (1000 ml) and hydrochloric acid (25 ml) was stirred for 3 hrs at 25-30° C. The solid obtained was filtered and washed with acetone. Acetone (200 ml) was added to the wet solid and stirred for 30 min. at reflux temperature. The reaction mixture was cooled to 25-30° C. and stirred for 1 hour. The solid formed was filtered, washed with acetone and dried to get the title compound.

Yield: 77 grams; M.R:198-203° C.

### Example 16

### Purification of 5,6,7,7a-tetrahydro-4H-thieno [3,2-c]-pyridin-2-one hydrochloride Compound of Formula-12b

50 gms of crude 5,6,7,7a-tetrahydro-4H-thieno [3,2-c]-pyridin-2-one hydrochloride compound of formula-12b obtained in example-15 is taken in methylene chloride (50 ml) and methanol (50 ml). Heated the reaction mixture to 40-45° C. and stirred for 30 min at same temperature. Cooled the reaction mixture to 25-30° C. and stirred for 1 hour. Filtered the solid and washed with methylene chloride. Dried the compound to get the pure title compound

Yield: 45 gms.

### Example 17

### Purification of 5,6,7,7a-tetrahydro-4H-thieno [3,2-c]-pyridin-2-one hydrochloride Compound of Formula-12b

50 gms of crude 5,6,7,7a-tetrahydro-4H-thieno [3,2-c]-pyridin-2-one hydrochloride compound of formula-12b obtained in example-15 is taken in methylene chloride (50 ml) and methanol (50 ml) and dimethyl formamide (25 ml). Heated the reaction mixture to 40-45° C. and stirred for 30 min at same temperature. Cooled the reaction mixture to 25-30° C. and stirred for 1 hour. Filtered the solid and washed with methylene chloride. Dried the compound to get the pure title compound

Yield: 44 gins; MR: 203-206° C.

### Example 18

### Preparation of 2-bromo-1-cyclopropyl-2-(2-fluorophenyl)ethanone Compound of Formula-3

A mixture of 1-cyclopropyl-2-(2-fluorophenyl) ethanone (40 grams), chloroform (400 ml), N-bromo succinamide (50 grams), azobisisobutyronitrile (2.4 grams) and p-toluenesulfonic acid (1.2 grams) was stirred for 4 hrs at reflux temperature. The reaction mixture was cooled to 0-5° C. and stirred for 45 minutes and the precipitated solid was filtered. The filtrate was distilled off completely under reduced pressure. Cyclohexane (100 ml) was added to the obtained residue and distilled off the solvent under reduced pressure to get the title compound.

Yield: 55 grams

### Example 19

### Preparation of 5-(α-cyclopropylcarbonyl-2-fluorobenzyl)-2-oxo-2,4,5,6,7,7a-hexahydro thieno[3,2-c] pyridine Compound of Formula-7

A mixture of 5,6,7,7a-tetrahydro-4H-thieno[3,2-c]-pyridin-2-one-hydrochloride (100 grams), potassium carbonate (63.5 grams) and acetonitrile (1000 ml) was stirred for 30 minutes at 25-30° C. 2-bromo-1-cyclopropyl-2-(2-fluorophenyl) ethanone (66 grams) in acetonitrile (30 ml) was added to the reaction mixture and stirred for 7 hours at 25-30° C. The reaction mixture was filtered and removed the precipitated solid. The filtrate was distilled off completely under reduced pressure; ethyl acetate followed by cyclohexane was added to it. The reaction mixture was stirred for 25 minutes at 40-45° C. The reaction mixture was cooled to 25-30° C. and stirred for an hour. The reaction mixture was filtered and solvent from the filtrate was distilled off completely under reduced pressure to get the title compound.

Yield: 70 grams

### Example 20

### Preparation of 5-(α-cyclopropylcarbonyl-2-fluorobenzyl)-2-oxo-2,4,5,6,7,7a-hexahydro thieno[3,2-c] pyridine hydrobromide

A mixture of 5,6,7,7a-tetrahydro-4H-thieno[3,2-c]pyridin-2-one-p-toluene sulfonate (100 grams), potassium carbonate (63.5 grams) and acetonitrile (1000 ml) was stirred for 30 minutes at 25-30° C. 2-bromo-1-cyclopropyl-2-(2-fluorophenyl) ethanone (66 grams) in acetonitrile (30 ml) was added to the reaction mixture and stirred for 7 hours at 25-30° C. The reaction mixture was filtered and removed the precipitated solid. The filtrate was distilled off completely under reduced pressure; ethyl acetate followed by cyclohexane was added to it. The reaction mixture was stirred for 25 minutes at 40-45° C. The reaction mixture was cooled to 25-30° C. and stirred for an hour. The reaction mixture was filtered and solvent from the filtrate was distilled off completely under reduced pressure. 250 ml of acetone was added to the obtained compound and cooled the reaction mixture to 0-5° C. Aqueous hydro bromide (70 ml) was slowly added to the reaction mixture. Raised the temperature to 20-25° C. and stirred for 6 hrs at same temperature. Cooled the reaction mixture to 0-5° C. and stirred for 2 hrs at same temperature. Filtered the precipitated solid and washed with acetone to get the title compound.

Yield: 85 gms.

### Example 21

### Preparation of 5-(α-cyclopropylcarbonyl-2-fluorobenzyl)-2-oxo-2,4,5,6,7,7a-hexahydro thieno[3,2-c] pyridine hydrochloride

100 gms of 5-(α-cyclopropylcarbonyl-2-fluorobenzyl)-2-oxo-2,4,5,6,7,7a-hexahydro thieno[3,2-c] pyridine hydro bromide was taken in methylene chloride(300 ml) and water (500 ml) and cooled the reaction mixture to 0-5° C. Basifying the reaction mixture with aqueous ammonia(25 ml). Raised the temperature to 25-30° C. and separated the both aqueous and organic layers. Washed the organic layer with water and distilled off the solvent completely under reduced pressure. Co-distilled the compound with acetone. 800 ml of acetone was added to the obtained compound and cooled the reaction mixture to 25-30° C. Ethyl acetate hydrochloride(200 ml) was added to the obtained compound and stirred for 1 hour. Filtered the precipitated solid and dried the compound.

Yield: 80 gms; MR: 178-183° C.

### Example 22

### Preparation of 5-(α-cyclopropylcarbonyl-2-fluorobenzyl)-2-oxo-2,4,5,6,7,7a-hexahydro thieno[3,2-c] pyridine hydrochloride

A mixture of 5,6,7,7a-tetrahydro-4H-thieno[3,2-c]-pyridin-2-one-hydrochloride (100 grams), potassium carbonate (63.5 grams) and acetonitrile (1000 ml) was stirred for 30 minutes at 25-30° C. 2-bromo-1-cyclopropyl-2-(2-fluorophenyl) ethanone (66 grams) in acetonitrile (30 ml) was added to the reaction mixture and stirred for 7 hours at 25-30° C. The reaction mixture was filtered and removed the precipitated solid. The filtrate was absorbed with silica gel and passed the material through silica gel bed using, ethyl acetate and cyclohexane in 1:1 ratio. Distilled the solvent completely under reduced pressure. 650 ml of acetone was added to the obtained compound and cooled the reaction mixture to 25-30° C. Ethyl acetate hydrochloride(175 ml) was added to the obtained compound and stirred for 1 hour. Filtered the precipitated solid and dried the compound.

Yield: 65 gms; MR: 178-183° C.

### Example 23

### Preparation of prasugrel Compound of Formula-1

Triethylamine (32 grams) was added to the solution of 5-(α-cyclo propylcarbonyl-2-fluorobenzyl)-2-oxo-2,4,5,6,7,7a-hexahydrothieno[3,2-c] pyridine (60 grams) in toluene (360 ml) and stirred for 15 minutes at 25-30° C. and then cooled to 0-5° C. Acetic anhydride (64.5 ml) was added to the reaction mixture and stirred for 30 minutes at 0-5° C. The reaction mixture was stirred for 6 hours at 25-30° C. The reaction mixture was quenched with water and separated both the aqueous and organic layers. Organic layer washed with aqueous sodium bicarbonate followed by water and then the solvent from the organic layer was distilled off under reduced pressure and methanol (75 ml) was added to the obtained residue and stirred for 45 min at reflux temperature. The reaction mixture was cooled to 0-5° C. and stirred for 45 minutes. The obtained solid was filtered, washed with methanol and then dried to get the title compound.

Yield: 36 grams; Purity by HPLC: 99.00%

### Example 24

### Preparation of prasugrel Compound of Formula-1

Triethylamine (32 grams) was added to the solution of 5-(α-cyclo propylcarbonyl-2-fluorobenzyl)-2-oxo-2,4,5,6,7,7a-hexahydrothieno[3,2-c] pyridine hydrobromide (60 grams) in methylene chloride (600 ml) and stirred for 15 minutes at 25-30° C. and then cooled to 0-5° C. Acetic anhydride (64.5 ml) was added to the reaction mixture and stirred for 6 hrs at 0-5° C. Water was added to the reaction mixture and raised the temperature to 20-25° C. Separated both the aqueous and organic layers. Organic layer washed with aqueous sodium bicarbonate followed by water and then the solvent from the organic layer was distilled off under reduced pressure, isopropyl alcohol (48 ml) and acetonitrile (16 ml) was added to the obtained residue and stirred for 45 min at reflux temperature. The reaction mixture was cooled to 10-15° C. and stirred for 60 minutes. The obtained solid was filtered, washed with isopropyl alcohol and then dried to get the title compound.

Yield: 45 grams; Purity by HPLC: 99.20%

### Example 25

### Preparation of crystalline form-M of 5-(α-cyclopropylcarbonyl-2-fluorobenzyl)-2-oxo-2,4,5,6,7,7a-hexahydro thieno[3,2-c] pyridine hydrobromide

Acetone (130 ml) was added to 5-(α-cyclopropylcarbonyl-2-fluorobenzyl)-2-oxo-2,4,5,6,7,7a-hexahydro thieno[3,2-c] pyridine obtained in example-16 (55 grams) at 25-30° C. The reaction mixture was cooled to 0-5° C. and aqueous hydrobromide (16 grams) was added to it and stirred for an hour at 0-5° C. The obtained solid was filtered, washed with acetone and dried to get the title compound.

Yield: 30 grams; Purity by HPLC: 98.42%

### Example-26

### Preparation, of crystalline form-S of 5-(α-cyclopropylcarbonyl-2-fluorobenzyl)-2-oxo-2,4,5,6,7,7a-hexahydro thieno[3,2-c] pyridine

A mixture of 5-(α-cyclopropylcarbonyl-2-fluorobenzyl)-2-oxo-2,4,5,6,7,7a-hexahydro thieno[3,2-c] pyridine hydrobromide (50 grams), water (500 ml) and 10% sodium bicarbonate solution (500 ml) was stirred for 30 minutes at 10-15° C. Methylene chloride (500 ml) was added to the reaction mixture and stirred for 15 min at 25-30° C. The organic and aqueous layer was separated and the solvent from the organic layer was distilled off completely under reduced pressure. Cyclohexane (150 ml) was added to the residue and stirred the reaction mixture for 1 hour at 25-30° C. The obtained solid was filtered, washed with cyclohexane and dried to get the title compound as crystals.

Yield: 40 grams; Purity by HPLC: 99.00%

### Example 27

### Preparation of Prasugrel hydrochloride

A mixture of 5-(α-cyclopropylcarbonyl-2-fluorobenzyl)-2-oxo-2,4,5,6,7,7a-hexahydro thieno[3,2-c] pyridine (100 grams), acetone (1000 ml) and hydrochloric acid (10.5 grams) was stirred for 3 hours at 25-30° C. The precipitated solid was filtered and washed with acetone. To the wet material added acetone (300 ml) and stirred for 1 hour at 25-30° C. Filtered the reaction mixture, washed with acetone and dried the compound to get the title compound.

Yield: 47 grams;

M.R: 197-202° C.; Purity: 99.90% by HPLC

### Example 28

### Preparation of Prasugrel hydrochloride

A mixture of 5-(α-cyclopropylcarbonyl-2-fluorobenzyl)-2-oxo-2,4,5,6,7,7a-hexahydro thieno[3,2-c] pyridine (100 grams) and acetone (800 ml) was heated to 40-45° C. and the obtained solution was subjected to carbon treatment and then filtered. Filtrate was cooled to 0-5° C. and 2.5% ethyl acetate hydrochloric acid (250 ml) was added to it and stirred for 45 minutes. The solid obtained was filtered, washed with acetone and dried the compound to get the title compound.

Yield: 90 grams; Purity: 99.91% by HPLC

### Example 29

### One Pot Process for the Preparation of Prasugrel

A mixture of 5,6,7,7a-tetrahydro-4H-thieno[3,2-c]-pyridin-2-one hydrochloride (100 grams), potassium carbonate (215 grams) and acetonitrile (500 ml) was stirred for 30 minutes at 25-30° C. 2-bromo-1-cyclopropyl-2-(2-fluorophenyl)ethanone (89.5 grams) in acetonitrile (50 ml) was added to the reaction mixture and stirred for 6 hours at 25-30° C. The reaction mixture was filtered and removed the precipitated solid. Triethylamine (98.2 grams) was added to the filtrate and then cooled to 0-5° C. Acetic anhydride (119 grams) was added to the reaction mixture and stirred for 30 minutes at 0-5° C. The reaction mixture was stirred for 6 hours at 25-30° C. Then the reaction mixture was quenched with water and extracted the reaction mixture into toluene. The solvent from the toluene layer was distilled off under reduced pressure and methanol (100 ml) was added to the obtained residue and stirred for 45 min at reflux temperature. The reaction mixture was cooled to 0-5° C. and stirred for 45 minutes. The obtained solid was filtered, washed with methanol and then dried to get the title compound.

Yield: 60 grams; Purity by HPLC: 98.97%

### Example 30

### Preparation of 2-(2-fluorophenyl)-N-methoxy-N-methylacetamide Compound of General Formula-15a

A mixture of N,O-dimethyl hydroxyl amine hydrochloride (76 grams) and methylene chloride (1000 ml) was cooled to 0-5° C. Triethyl amine (132 grams) was added to the reaction mixture and stirred for 15 minutes. 1-hydroxybenzotriazole (HOBt) (8.75 grams) was added to the reaction mixture followed by 2-(2-fluorophenyl acetic acid) (100 grams) at 0-5° C. Stirred the reaction mixture for 5 minutes and then added the mixture of dicyclohexylcarbodiimide (DCC) (134 grams) in 150 ml of methylene chloride to the reaction mixture at 0-5° C. and stirred for 6 hours. The obtained solid was filtered off and washed with methylene chloride. The filtrate was washed with water. The organic layer was washed with aq.hydrochloric acid followed by water then sodium bicarbonate solution followed by water. Distilled off the solvent from the organic layer under reduced pressure. Methylene chloride was added to the obtained residue at 25-30° C. and stirred for up to dissolution. The reaction mixture was stirred at 0-5° C. for 45 minutes and the obtained solid was removed by filtration. The filtrate was distilled off completely under reduced pressure. Ether was added to the obtained residue, heated to reflux and then stirred the reaction mixture for 20 minutes at the same temperature. The reaction mixture was cooled to 0-5° C. and stirred for 60 minutes. The solid obtained was filtered, washed with ether and dried to get the title compound.

Yield: 108 grams.

### Example 31

### Preparation of 1-cyclopropyl-2-(2-fluorophenyl) ethanone Compound of Formula-16

Cyclopropyl bromide (122.5 grams) was added to the suspension of magnesium (24.5 grams) in tetrahydrofuran (700 ml) and iodine (0.03 grams) at 25-30° C. then the reaction mixture was heated to 40-50° C. The mixture of 2-(2-fluorophenyl)-N-methoxy-N-methylacetamide (100 grams) and tetrahydrofuran (300 ml) was added to the reaction mixture and stirred at 40-50° C. After completion of the reaction, the reaction mixture was cooled to 0-5° C. and quenched it with aq. hydrochloric acid. Stirred the reaction mixture for 15 minutes at 25-30° C. and the reaction mixture was extracted with ethyl acetate. The organic layer was washed with aq. sodium chloride solution followed by water. The ethyl acetate layer was distilled off under reduced pressure to get the title compound.

Yield: 97 grams

### Example 32

### Preparation of 2-bromo-1-cyclopropyl-2-(2-fluorophenyl) ethanone Compound of Formula-3

A mixture of 1-cyclopropyl-2-(2-fluorophenyl) ethanone (40 grams), methylene chloride (400 ml), N-bromo succinamide (50 grams), azobisisobutyronitrile (2.4 grams) and p-toluenesulfonic acid (1.2 grams) was stirred for 4 hrs at reflux temperature. The reaction mixture was cooled to 0-5° C. and stirred for 45 minutes and the precipitated solid was filtered. The filtrate was distilled off completely under reduced pressure. Cyclohexane (100 ml) was added to the obtained residue and distilled off the solvent under reduced pressure to get the title compound.

Yield: 55 grams

### Example 33

### Purification of 1-cyclopropyl-2-(2-fluorophenyl) ethanone Compound of Formula-16

The crude 1-cyclopropyl-2-(2-fluorophenyl) ethanone compound of formula-5 (100 grams) (having purity of 93.41% and containing 3.73% of 1-methyl-2-(2-fluoro phenyl)ethanone) prepared as per the reported process was charged into a clean and dry vessel and was purified by fraction distillation. The main fraction was collected at a vapour temperature of 80-90° C. under reduced pressure to get 82 grams of the pure title compound.

Purity by GC: 98.53%; 1-methyl-2-(2-fluorophenyl)ethanone: 0.02%

### Example 34

### Preparation of 2-bromo-1-cyclopropyl-2-(2-fluorophenyl) ethanone Compound of Formula-3

The title compound was prepared in a similar manner to example-32 except that pure compound of formula-16 obtained as per example-33 is used as a input in place of 1-cyclopropyl-2-(2-fluorophenyl) ethanone.

Yield: 56 grams

### Example 35

### Preparation of 5-(α-cyclopropylcarbonyl-2-fluorobenzyl)-2-oxo-2,4,5,6,7,7a-hexahydro thieno[3,2-c] pyridine compound of formula-7

A mixture of 5,6,7,7a-tetrahydro-4H-thieno[3,2-c]-pyridin-2-one-p-toluene sulfonate (100 grams), potassium carbonate (63.5 grams) and acetonitrile (1000 ml) was stirred for 30 minutes at 25-30° C. 2-bromo-1-cyclopropyl-2-(2-fluorophenyl) ethanone (66 grams) prepared as per example-34 in acetonitrile (30 ml) was added to the reaction mixture and stirred for 7 hours at 25-30° C. The reaction mixture was filtered and removed the precipitated solid. The filtrate was distilled off completely under reduced pressure, ethyl acetate followed by cyclohexane was added to it. The reaction mixture was stirred for 25 minutes at 40-45° C. The reaction mixture was cooled to 25-30° C. and stirred for an hour. The reaction mixture was filtered and solvent form the filtrated was distilled off completely under reduced pressure to get the title compound.

Yield: 70 grams

### Example 36

### Preparation of 5-[(1RS)-2-cyclopropyl-1-(2-fluorophenyl)-2-oxoethyl]-4,5,6,7-tetrahydrothieno[3,2-c]pyridin-2-yl acetate Compound of Formula-1

Triethylamine (98 grams) was added to a solution of 5-(α-cyclopropylcarbonyl-2-fluorobenzyl)-2-oxo-2,4,5,6,7,7a-hexahydrothieno[3,2-c]pyridine (100 grams) prepared as per example-35 in methylene chloride (1000 ml) and stirred for 15 minutes at 25-30° C. The reaction mixture was cooled to 0-5° C. and acetic anhydride (62 grams) was added to it and then stirred for 6 hrs at 0-5° C. Added water (300 ml) to the reaction mixture at 25-30° C. and stirred for 15 minutes. Separated the both aqueous and organic layers and the organic layer was washed with aq.sodium bicarbonate solution followed by washed with water. Distilled of the solvent from organic layer completely under reduced pressure. Added 75 ml of cyclohexane to the reaction mixture and distilled off the solvent completely under reduced pressure. To the obtained solid added acetonitrile (50 ml) at 25-30° C. and heated the reaction mixture to 40-50° C. Stirred the reaction mixture for 20 minutes at same temperature. Added isopropyl alcohol (75 ml) to the reaction mixture at 25-30° C. and then heated to 40-50° C. Stirred the reaction mixture for 30 minutes at same temperature. Cooled the reaction mixture to 10-15° C. and stirred for 1 hr at same temperature. Filtered the precipitated solid and washed with isopropyl alcohol. Dried the compound to get the highly pure title compound.

Yield: 75 grams; Melting point: 120-125° C.

Purity by HPLC: 99.55%; Methyl keto impurity: Not detected

### Example 37

### Preparation of 5-[(1RS)-2-cyclopropyl-1-(2-fluorophenyl)-2-oxoethyl]-4,5,6,7-tetrahydrothieno[3,2-c]pyridin-2-yl acetate hydrochloride Compound of Formula-1a

A mixture of 5-[(1RS)-2-cyclopropyl-1-(2-fluorophenyl)-2-oxoethyl]-4,5,6,7-tetrahydrothieno[3,2-c]pyridin-2-yl acetate(100 -grams) prepared as per example-36 and acetone(900 ml) was heated to 30-40° C. Added 5 grams of activated carbon to the above reaction mixture at same temperature and stirred for 15 minutes. Filtered the reaction mixture and the filtrate was heated to 50-55° C. under nitrogen atmosphere. Added slowly 500 ml of ethyl acetate hydrochloride solution to the reaction mixture at 50-55° C. Stirred the reaction mixture for 45 minutes at 25-30° C. under nitrogen atmosphere. Filtered the precipitated solid and washed with acetone. Acetone(1000 ml) was added to the obtained compound and stirred for 45 minutes at 25-30° C. under nitrogen atmosphere. Filtered the precipitated solid and washed with acetone. Dried the compound to get the highly pure title compound.

Yield: 83 grams.

Purity by HPLC: 99.78% ; PSD: D_{[4,3]}=62.204μ; D₁₀=2.330μ, D₅₀=50.777μ; D₉₀=117.203μ.

### Example 38

### Preparation of 5-[(1RS)-2-cyclopropyl-1-(2-fluorophenyl)-2-oxoethyl]-4,5,6,7-tetrahydrothieno[3,2-c]pyridin-2-yl acetate hydrochloride Compound of Formula-1a

A mixture of 5-[(1RS)-2-cyclopropyl-1-(2-fluorophenyl)-2-oxoethyl]-4,5,6,7-tetrahydrothieno[3,2-c]pyridin-2-yl acetate(100 grams) prepared as per example-36 and acetone(900 ml) was heated to 30-40° C. Added 5 grams of activated carbon to the above reaction mixture at same temperature and stirred for 15 minutes. Filtered the reaction mixture and the filtrate was heated to 30-40° C. under nitrogen atmosphere. Added slowly 500 ml of ethyl acetate hydrochloride solution to the reaction mixture at 20-30° C. Stirred the reaction mixture for 4 hrs. at 25-30° C. under nitrogen atmosphere. Filtered the precipitated solid and washed with acetone. Acetone(1000 ml) was added to the obtained compound and stirred for 6 hrs at 25-30° C. under nitrogen atmosphere. Filtered the precipitated solid and washed with acetone. Dried the compound to get the highly pure title compound.

Yield: 83 grams.

Purity by HPLC: 99.78%; PSD: D_{[4,3]}=58.57882 ; D₁₀=2.807μ, D₅₀=44.266μ; D₉₀=127.775μ.

### Example 39

### Purification of 5-[(1RS)-2-cyclopropyl-1-(2-fluorophenyl)-2-oxoethyl]-4,5,6,7-tetrahydrothieno[3,2-c]pyridin-2-yl acetate Compound of Formula-1

To the crude 5-[(1RS)-2-cyclopropyl-1-(2-fluorophenyl)-2-oxoethyl]-4,5,6,7-tetrahydrothieno[3,2-c]pyridin-2-yl acetate (50 grams), acetonitrile (25 ml) was added and heated to 40-50° C. The reaction mixture was stirred for 20 min at 40-50° C. Isopropyl alcohol (40 ml) was added to the reaction mixture at 25-30° C. and heated to 40-50° C. then stirred for 30 minutes. The reaction mixture was cooled to 10-15° C. and stirred for 1 hr. The precipitated solid was filtered and washed with isopropyl alcohol and then dried to get the highly pure title compound.

Yield: 42 grams, Purity by HPLC: 99.61%

### Example 40

### Purification of Prasugrel

A mixture of prasugrel (50 grams) and isopropyl alcohol (75 ml) and acetonitrile (25 ml) was heated to 50-55° C. The reaction mixture was stirred for an hour at same temperature. Cooled the reaction mixture to 10-15° C. and filtered the precipitated solid. Washed with isopropyl alcohol and then dried to get high pure prasugrel.

Yield: 45 grams; Purity: 99.90% by HPLC.

### Example 41

### Purification of Prasugrel

A mixture of prasugrel (50 grams) and acetonitrile (100 ml) was heated to 60-65° C. The reaction mixture was stirred for an hour at same temperature. Cooled the reaction mixture to 0-5° C. and filtered the precipitated solid. Washed with chilled acetonitrile and then dried to get high pure prasugrel.

Yield: 40 grams; Purity: 99.91% by HPLC.

1. A novel process for the preparation of Prasugrel and its pharmaceutically acceptable salts; comprising:
a) Reacting the 4,5,6,7-tetrahydrothieno[3,2-c] pyridine compound of formula-2 or its salts, 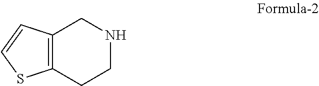
with α-cyclopropylcarbonyl-2-fluorobenzyl bromide compound of formula-3, 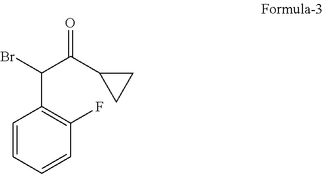
in presence of a suitable base in a suitable solvent to provide 5-(α-cyclopropylcarbonyl-2-fluorobenzyl)-4,5,6,7,-tetrahydrothieno[3,2-c] pyridine compound of formula-4, 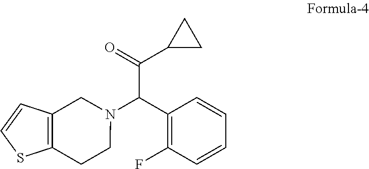
b) converting the 5-(α-cyclopropylcarbonyl-2-fluorobenzyl)-4,5,6,7,-tetrahydro thieno[3,2-c] pyridine compound of formula-4 into 5-(α-cyclopropylcarbonyl-2-fluorobenzyl)-2-oxo-2,4,5,6,7,7a-hexahydro thieno[3,2-c] pyridine compound of Formula-7 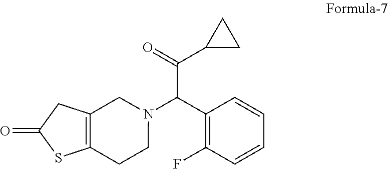
by in-situ protecting the keto functionality of compound of formula-4 as an enolate by treating with a lithium reagent and by introducing a boronic group —B(OR′)₂ at second position of thieno[3,2-c] pyridine skeleton by treating it with second lithium reagent in a suitable solvent and a suitable boronating agent, in presence or absence of co-solvent and subsequent oxidation by treating it with suitable oxidizing agent to provide compound of Formula-7,
c) acetylating the compound of Formula-7 with a suitable acetylating agent in a suitable solvent in presence of a suitable organic base selected to provide the compound of formula-1, 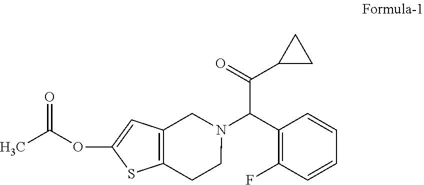
d) optionally converting the prasugrel into its acid addition salts by treating it with a suitable acid in a suitable solvent to provide an acid addition salt of prasugrel.

2. The process according to claim 1, wherein;
in step a) the suitable base is selected from a group consisting of alkali metal carbonates like sodium carbonate, potassium carbonate; or an alkali metal hydroxide like sodium hydroxide, potassium hydroxide; or alkali metal bicarbonates like sodium bicarbonate, potassium bicarbonate; alkali metal alkoxides like sodium tertiary butoxide, potassium tertiary butoxide or an organic base like triethylamine, tributylamine, diisopropylethlyamine preferably potassium carbonate, in a suitable solvent selected from aliphatic hydrocarbons like hexane, cyclohexane, petroleum ether; or aromatic hydrocarbons like xylene, toluene; or halogenated hydrocarbons like dichloromethane, chloroform, 1,2-dichloroethane; or ethers like diethyl ether, diisopropyl ether, tetrahydrofuran, dimethoxy ethane; or ketones like acetone, methyl ethyl ketone, diethyl ketone; or acetates like ethyl acetate, propyl acetate, butyl acetate; alcohols like methanol, ethanol, propanol, butanol, isopropanol; or nitriles like acetonitrile and propionitrile; dimethyl formamide, dimethyl acetamide and dimethyl sulfoxide or mixtures thereof preferably acetonitrile;
in step b) the suitable lithium derivative for protecting the keto functionality as enolate is selected from n-butyl lithium, sec-butyl lithium, tert-butyl lithium lithium hexamethyldisilazide and lithium diisopropylamide preferably lithium diisopropylamide; the suitable boronating agent is selected from boron oxides such as B₂O₃, boron acids such as H₃BO₃, lower alkyl esters of boron acids such as trimethylborate, triethylborate, tri n-butylborate, boron halides like BF₃, BCl₃, salts of boron acids like sodium borate, ammonium borate preferably tri n-butylborate; the suitable lithiating agent is selected from n-butyl lithium, sec-butyl lithium, tert-butyl lithium lithium hexamethyldisilazide and lithium diisopropylamide preferably n-butyl lithium; the suitable oxidising agent is selected from nitric acid, hydrogen peroxide, per acids such as peracetic acid, trifluoro peracetic acid, perbenzoic acid, m-chloro perbenzoic acid and the like; ozone, manganese dioxide, potassium permanganate, chromic acid, chromium trioxide, selenium dioxide, sodium hypochlorite, sodium metaperiodate and the like, preferably hydrogen peroxide; the suitable co solvent is selected from tetramethyl urea(TMU), 1,3-Dimethyl-3,4,5,6-tetrahydro-2(1H)-pyrimidinone (DMPU), N-Methyl-2-pyrrolidone (NMP), hexamethylphosphoramide (HMPA) and the like; the suitable solvent is selected from aliphatic hydrocarbons like hexane, cyclohexane, petroleum ether; or aromatic hydrocarbons like xylene, toluene; or halogenated hydrocarbons like dichloromethane, chloroform, 1,2-dichloroethane; or ethers like diethyl ether, diisopropyl ether, tetrahydrofuran, dimethoxy ethane; or ketones like acetone, methyl ethyl ketone, diethyl ketone; or acetates like ethyl acetate, propyl acetate, butyl acetate; alcohols like methanol, ethanol, propanol, butanol, isopropanol; or nitriles like acetonitrile and propionitrile; dimethyl formamide, dimethyl acetamide and dimethyl sulfoxide or mixtures thereof preferably tetrahydrofuran;
in step c) the suitable acetylating agent is like acetic anhydride in a suitable solvent selected from diethylether, tetrahydrofuran, dioxane, acetone, methylethyl ketone, ethyl acetate, acetonitrile, dimethyl formamide, dimethyl acetamide and dimethyl sulfoxide preferably acetonitrile, in presence of a suitable organic base selected from triethyl amine, tributyl amine, pyridine, 4-dimethylaminopyridine, N-methylmorpholine and diisopropylethyl amine preferably triethylamine; and
in step d) the suitable acid selected from an inorganic acids such as hydrochloric acid, hydrobromic acid; or an organic acids such as benzene sulfonic acid, maleic acid, oxalic acid, fumaric acid, succinic acid, p-tolunesulfonic acid and malic acid, in a suitable solvent selected from aliphatic hydrocarbons like hexane, cyclohexane, petroleum ether;
or aromatic hydrocarbons like xylene, toluene; or halogenated hydrocarbons like dichloromethane, chloroform, 1,2-dichloroethane; or ethers like diethyl ether, diisopropyl ether, tetrahydrofuran, dimethoxy ethane; or ketones like acetone, methyl ethyl ketone, diethyl ketone; or acetates like ethyl acetate, propyl acetate, butyl acetate; alcohol like isopropyl alcohol; or nitriles like acetonitrile and propionitrile or mixtures thereof.

3. A novel process for the preparation of prasugrel hydrochloride of Formula-1a, 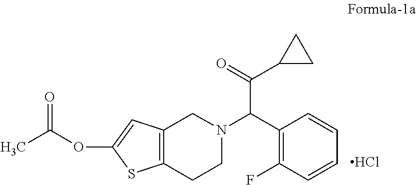
comprising:
a) Reacting the 4,5,6,7-tetrahydrothieno[3,2-c] pyridine hydrochloride compound of formula-2a 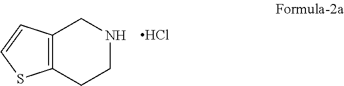
with α-cyclopropylcarbonyl-2-fluorobenzyl bromide compound of formula-3, 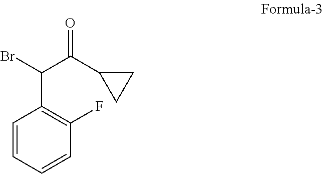
in presence of sodium carbonate in acetonitrile provides 5-(α-cyclopropylcarbonyl-2-fluorobenzyl)-4,5,6,7,-tetrahydrothieno[3,2-c] pyridine compound of formula-4, 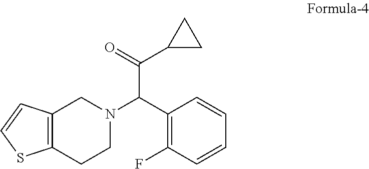
b) converting the 5-(α-cyclopropylcarbonyl-2-fluorobenzyl)-4,5,6,7,-tetrahydro thieno[3,2-c] pyridine compound of formula-4 into 5-(α-cyclopropylcarbonyl-2-fluorobenzyl)-2-oxo-2,4,5,6,7,7a-hexahydrothieno[3,2-c]pyridine compound of Formula-7, 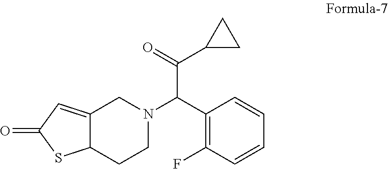
by in-situ protecting the keto functionality of compound of formula-4 as an enolate by treating with a lithium reagent and by introducing a boronic group —B(OR′)₂ at second position of thieno[3,2-c] pyridine skeleton by treating it with second lithium reagent in a suitable solvent and a suitable boronating agent, in presence or absence of co-solvent and subsequent oxidation by treating it with suitable oxidizing agent to provide compound of Formula-7,
c) acetylating the compound of formula-7 with acetic anhydride in presence of triethyl amine in acetonitrile to provide prasugrel compound of formula-1, 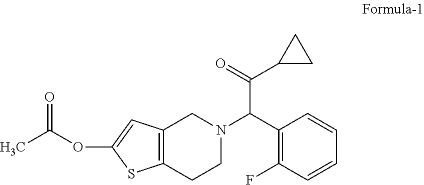
d) treating the prasugrel with hydrochloric acid in a suitable solvent to provide prasugrel hydrochloride compound of formula-1a.

4. Acid addition salts of 5-(α-cyclopropylcarbonyl-2-fluorobenzyl)-4,5,6,7,-tetrahydro thieno[3,2-c] pyridine compounds of general Formula-8, with the proviso that the acid addition salt is not hydrochloride

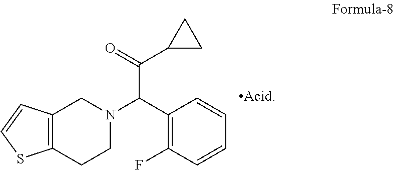

5. The acid addition salt according to claim 4, wherein the acid is selected from an inorganic acids such as hydrobromic acid, sulfuric acid, nitric acid or an organic acids such as benzene sulfonic acid, maleic acid, oxalic acid, fumaric acid, succinic acid, p-tolunesulfonic acid and malic acid.

6. A process for the preparation of 5-(α-cyclopropylcarbonyl-2-fluorobenzyl)-2-oxo-2,4,5,6,7,7a-hexahydrothieno[3,2-c]pyridine compound of Formula-7, 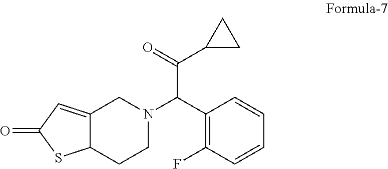
comprising reacting the 5-(α-cyclopropylcarbonyl-2-fluorobenzyl)-4,5,6,7,-tetrahydrothieno [3,2-c]pyridine compound of formula-4, 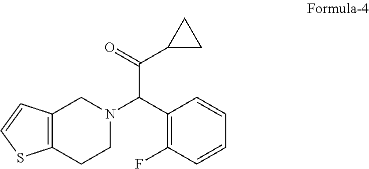
with lithium reagent for in-situ protection the keto functionality as enolate, followed by introduction of a boronic group —B(OR′)₂ at second position of thieno[3,2-c] pyridine skeleton by treating it with second lithium reagent in a suitable solvent and a suitable boronating agent, in presence or absence of co-solvent and subsequent oxidation by treating it with suitable oxidizing agent to provide compound of Formula-7.

7. A process for the preparation of 5-(α-cyclopropylcarbonyl-2-fluorobenzyl)-2-oxo-2,4,5,6,7,7a-hexahydrothieno[3,2-c]pyridine compound of Formula-7, 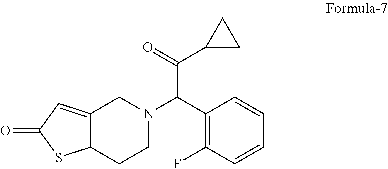
comprising reacting the 5-(α-cyclopropylcarbonyl-2-fluorobenzyl)-4,5,6,7,-tetra hydrothieno[3,2-c]pyridine compound of formula-4, 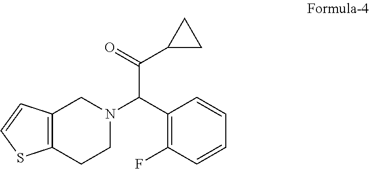
with lithium diisopropylamide for in-situ protection of keto functionality as enolate, followed by introduction a boronic group at second position of thieno[3,2-c] pyridine skeleton, by treating it with n-butyl lithium and then with tri n-butyl borate in tetrahydrofuran solvent, followed by treatment with hydrogen peroxide to provide 5-(α-cyclopropylcarbonyl-2-fluorobenzyl)-2-oxo-2,4,5,6,7,7a-hexahydrothieno[3,2-c] pyridine compound of Formula-7.

8. A process for the preparation of 5-(α-cyclopropylcarbonyl-2-fluorobenzyl)-2-oxo-2,4,5,6,7,7a-hexahydrothieno[3,2-c]pyridine compound of Formula-7, comprising:
a) Reacting the 4,5,6,7-tetrahydrothieno[3,2-c] pyridine hydrochloride compound of formula-2a, 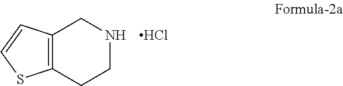
with α-cyclopropylcarbonyl-2-fluorobenzyl bromide compound of formula-3, 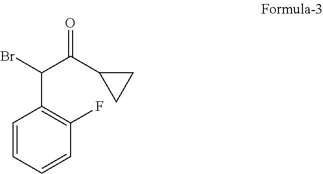
in presence of a suitable base in a suitable solvent to provide 5-(α-cyclopropylcarbonyl-2-fluorobenzyl)-4,5,6,7,-tetrahydrothieno[3,2-c] pyridine compound of formula-4, 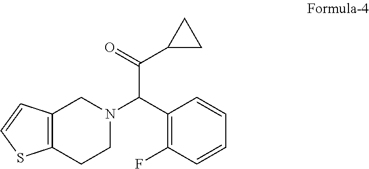
b) converting the 5-(α-cyclopropylcarbonyl-2-fluorobenzyl)-4,5,6,7,-tetrahydro thieno[3,2-c] pyridine compound of formula-4 into 5-(α-cyclopropylcarbonyl-2-fluorobenzyl)-2-oxo-2,4,5,6,7,7a-hexahydrothieno[3,2-c]pyridine compound of Formula-7 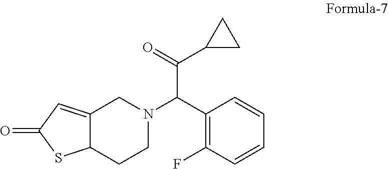
by in-situ protecting the keto functionality of compound of formula-4 as an enolate by treating with a lithium reagent and by introducing a boronic group —B(OR′)₂ at second position of thieno[3,2-c] pyridine skeleton by treating it with second lithium reagent in a suitable solvent and a suitable boronating agent, in presence or absence of co-solvent and subsequent oxidation by treating it with suitable oxidizing agent to provide compound of Formula-7.

9. A process for the preparation of 5-(α-cyclopropylcarbonyl-2-fluorobenzyl)-2-oxo-2,4,5,6,7,7a-hexahydrothieno[3,2-c]pyridine compound of Formula-7, comprising:
a) Reacting the 4,5,6,7-tetrahydrothieno[3,2-c] pyridine hydrochloride compound of formula-2a, 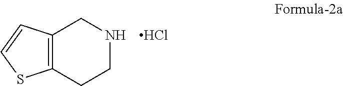
with a-cyclopropylcarbonyl-2-fluorobenzyl bromide compound of formula-3, 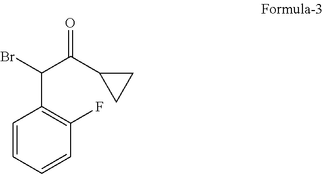
in presence of sodium carbonate in acetonitrile to provide 5-(α-cyclopropylcarbonyl-2-fluorobenzyl)-4,5,6,7,-tetrahydrothieno[3,2-c] pyridine compound of formula-4, 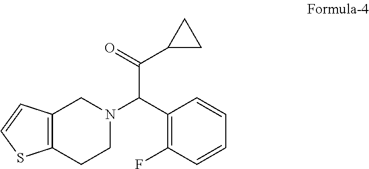
b) converting the 5-(α-cyclopropylcarbonyl-2-fluorobenzyl)-4,5,6,7,-tetrahydro thieno[3,2-c] pyridine compound of formula-4 into 5-(α-cyclopropylcarbonyl-2-fluorobenzyl)-2-oxo-2,4,5,6,7,7a-hexahydrothieno[3,2-c]pyridine compound of Formula-7 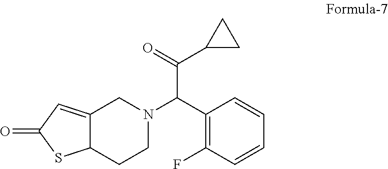
by in-situ protecting the keto functionality as enolate using lithium diisopropylamide and followed by introducing a boronic group —B(OR′)₂ at second position of thieno[3,2-c] pyridine skeleton, by treating it with n-butyl lithium and tri n-butyl borate in tetrahydrofuran, followed by treatment with hydrogen peroxide to provide 5-(α-cyclopropylcarbonyl-2-fluorobenzyl)-2-oxo-2,4,5,6,7,7a-hexahydro thieno[3,2-c] pyridine compound of Formula-7.

10. Use of acid addition salts of compounds of general Formula-8 as claimed in claim 4 in the preparation of highly pure 5-(α-cyclopropylcarbonyl-2-fluorobenzyl)-2-oxo-2,4,5,6,7,7a-hexahydrothieno[3,2-c] pyridine compound of Formula-7 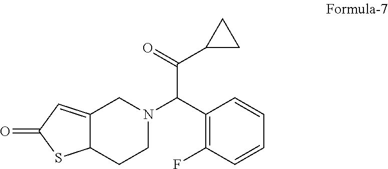
and prasugrel or its pharmaceutically acceptable salts.

11. Use of lithium diisopropyl amide for the protection of keto group in 5-(α-cyclopropylcarbonyl-2-fluorobenzyl)-4,5,6,7,-tetrahydrothieno[3,2-c] pyridine compound of formula-4

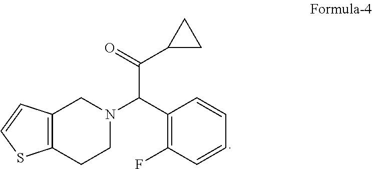

12. A salt of 5-(α-cyclopropylcarbonyl-2-fluorobenzyl)-2-oxo-2,4,5,6,7,7a-hexahydro thieno[3,2-c] pyridine having the following structural formula 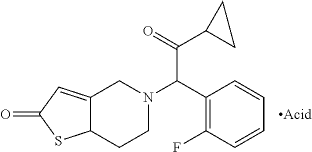
wherein “Acid” is an acid which is capable of forming acid addition salt with 5-(α-cyclopropylcarbonyl-2-fluorobenzyl)-2-oxo-2,4,5,6,7,7a-hexahydrothieno [3,2-c] pyridine and is selected from a group comprising of oxalic acid, succinic acid, fumaric acid, malonic acid, malic acid, maleic acid, d-tartaric acid, l-tartaric acid, dl-tartaric acid, citric acid, methanesulfonic acid, paratoluene sulfonic acid, acetic acid, sulfuric acid, phosphoric acid or hydrobromic acid, with a proviso that the acid is not hydrochloric acid.

13. A process for the preparation of a salt of 5-(α-cyclopropylcarbonyl-2-fluorobenzyl)-2-oxo-2,4,5,6,7,7a-hexahydro thieno[3,2-c] pyridine as claimed in claim 12, which comprises of treating the 5-(α-cyclopropylcarbonyl-2-fluoro benzyl)-2-oxo-2,4,5,6,7,7a-hexahydro thieno[3,2-c] pyridine with a suitable acid selected from oxalic acid, succinic acid, fumaric acid, malonic acid, malic acid, maleic acid, d-tartaric acid, l-tartaric acid, dl-tartaric acid, citric acid, methanesulfonic acid, paratoluene sulfonic acid, acetic acid, sulfuric acid, phosphoric acid and hydrobromic acid, in a suitable solvent selected from alcohols, ketones, esters, hydrocarbons, polar aprotic solvents, polar solvents, chloro solvents, nitriles or mixtures thereof for the sufficient period of time, to provide the corresponding salt of 5-(α-cyclopropylcarbonyl-2-fluorobenzyl)-2-oxo-2,4,5,6,7,7a-hexahydro thieno[3,2-c] pyridine.

14. A crystalline form-M of 5-(α-cyclopropylcarbonyl-2-fluorobenzyl)-2-oxo-2,4,5,6,7,7a-hexahydro thieno[3,2-c] pyridine hydrobromide, which is characterized by any one of the following;
a) Its Powder X-ray diffractogram having characteristic 2θ peaks at 7.07, 10.18, 14.81, 19.41, 20.44, 21.03, 22.37, 26.39, 26.86 and 27.32±0.2 degrees 2θ as illustrated in FIG. 1;
b) its Infra-Red spectrum having peaks at 3410.8, 3045.9, 2919.4, 2629.0, 2545.7, 1713.4, 1686.0, 1494.8, 1377.8, 1174.2, 1091.5, 1013.6, 1091.5 and 798.0 cm⁻¹ as illustrated in FIG. 2; and
c) Differential Scanning Calorimetry showing exothermic peak at 202.98° C. as illustrated in FIG. 3.

15. Use of a salt of 5-(α-cyclopropylcarbonyl-2-fluorobenzyl)-2-oxo-2,4,5,6,7,7a-hexahydrothieno[3,2-c] pyridine and crystalline 5-(α-cyclopropylcarbonyl-2-fluorobenzyl)-2-oxo-2,4,5,6,7,7a-hexahydrothieno [3,2-c] pyridine hydrobromide, in the preparation of highly pure 5-(α-cyclopropylcarbonyl-2-fluorobenzyl)-2-oxo-2,4,5,6,7,7a-hexahydro thieno [3,2-c] pyridine as well as in the preparation of highly pure prasugrel and its pharmaceutically acceptable salts.

16. Crystalline form-S of 5-(α-cyclopropylcarbonyl-2-fluorobenzyl)-2-oxo-2,4,5,6,7,7a-hexahydro thieno[3,2-c] pyridine, characterized by its Powder X-ray diffractogram having characteristic 2θ peaks at 7.63, 9.07, 12.73, 14.78, 15.30, 17.43, 18.20, 18.53, 19.47, 19.89, 21.70, 22.58, 24.00, 30.92±0.2 degrees 2θ as illustrated in FIG. 4.

17. A process for the purification/crystallization of 5-(α-cyclopropylcarbonyl-2-fluorobenzyl)-2-oxo-2,4,5,6,7,7a-hexahydrothieno[3,2-c] pyridine, comprising:
a) treating the crude 5-(α-cyclopropylcarbonyl-2-fluorobenzyl)-2-oxo-2,4,5,6,7,7a-hexahydro thieno[3,2-c] pyridine with a suitable acid as defined above, in a suitable solvent to provide the corresponding salt of 5-(α-cyclopropylcarbonyl-2-fluorobenzyl)-2-oxo-2,4,5,6,7,7a-hexahydrothieno [3,2-c] pyridine compound of formula-8, 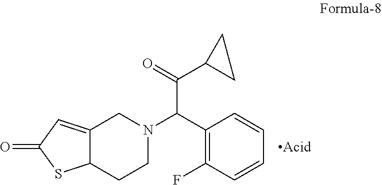
b) treating the thus obtained salt compound of formula-8 with a suitable organic or inorganic base, in a suitable solvent selected from alcohols, ketones, esters, hydrocarbons, polar aprotic solvents, polar solvents, chloro solvents, nitriles or mixtures thereof to provide pure crystalline 5-(α-cyclopropylcarbonyl-2-fluorobenzyl)-2-oxo-2,4,5,6,7,7a-hexahydro thieno[3,2-c] pyridine.

18. Crystalline form-N of prasugrel free base characterized by its Powder X-ray diffractogram having characteristic 2θ peaks at 7.80, 9.35, 11.79, 15.38, 15.64, 15.98, 16.28, 17.14, 18.78, 20.10, 20.36, 20.91, 21.35, 22.35, 22.63, 23.59, 24.39, 25.51, 29.43, 31.08 and 31.99±0.2 degrees 2θ as illustrated in FIG. 5.

19. A process for the preparation of prasugrel and its pharmaceutically acceptable salts, comprising acetylating 5-(α-cyclopropylcarbonyl-2-fluorobenzyl)-2-oxo-2,4,5,6,7,7a-hexahydro thieno[3,2-c] pyridine with a acetic anhydride in presence of a suitable organic base selected from triethyl amine, tributyl amine, pyridine, 4-dimethylaminopyridine, N-methylmorpholine and diisopropylethyl amine in a suitable hydrocarbon solvent or chloro solvent, followed by crystallization from a suitable alcoholic solvent to provide prasugrel compound of formula-1

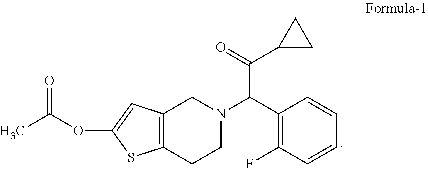

20. A process for the preparation of prasugrel and its pharmaceutically acceptable salts, especially hydrochloride salt, comprising:
a) protecting the amino functional group of 4,5,6,7-tetrahydrothieno[3,2-c] pyridine hydrochloride compound of formula-2a, 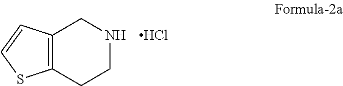
with triphenyl methyl chloride in presence of a suitable base like triethyl amine in a suitable solvent like methylene chloride, followed by crystallization from a suitable alcoholic solvents selected from methanol, ethanol, propanol, isopropyl alcohol and butanol or mixtures thereof, to provide a compound of formula-10, 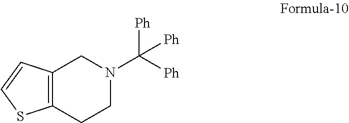
b) reacting the compound of formula-10 with n-butyl lithium in tetrahydrofuran at 0-5° C. under nitrogen atmosphere, followed by treating the reaction mixture with tri-n-butyl borate and then treating with hydrogen peroxide, followed by crystallization from a suitable alcoholic solvents like methanol, ethanol, propanol, isopropyl alcohol and butanol or mixtures thereof, to provide a compound of formula-11, 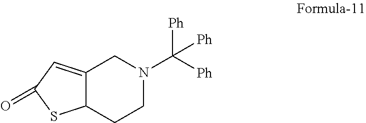
c) treating the compound of formula-11 with a suitable acid like hydrochloric acid or p-toluene sulfonic acid in a suitable solvent like acetone, followed by crystallization from acetone to provide the corresponding acid addition salt of 5,6,7,7a-tetrahydro-4H-thieno[3,2-c]pyridine-2-one compound of general formula-12, 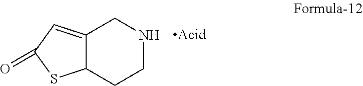
d) optionally purifying the compound of general formula-12 by treating with a suitable solvent or mixture of solvents,
e) reacting the 5,6,7,7a-tetrahydro-4H-thieno[3,2-c]pyridine-2-one acid addition salt compound of formula-12 or its free base with α-cyclopropylcarbonyl-2-fluorobenzyl bromide compound of formula-3, 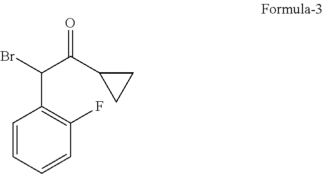
in presence of potassium carbonate in acetonitrile, followed by crystallization from a mixture of ethyl acetate and cyclohexane to provide compound of formula-7, 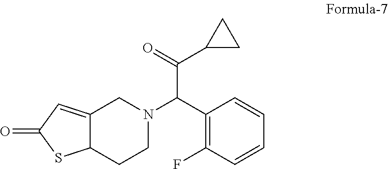
f) acetylating the compound of formula-7 with a suitable acetylating agent like acetic anhydride in a suitable solvent selected from hydrocarbon solvents like toluene, xylene, heptane, cyclohexane and hexane; chloro solvents like methylene chloride, chloroform and ethylene chloride in presence of a suitable organic base selected from triethyl amine, tributyl amine, pyridine, 4-dimethylaminopyridine, N-methylmorpholine and diisopropylethyl amine, followed by crystallization/isolation from a suitable alcoholic solvent, to provide the prasugrel compound of formula-1, 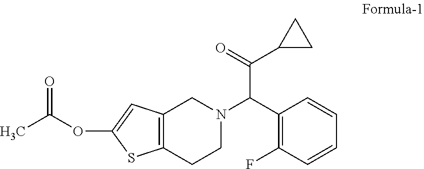
g) optionally converting the prasugrel into its hydrochloric acid salt by treating it with HCl gas or HCl gas dissolved in organic solvent like ethyl acetate, isopropyl acetate provides prasugrel hydrochloride salt compound of formula-1a

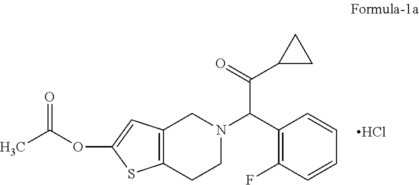

21. The process according to claim 20 wherein in step d) the solvent used for the purification is selected from chloro solvents, alcohol solvents, ester solvents, polar aprotic solvents or mixtures there of.

22. The process according to claim 20 wherein in step d) the solvent used for the purification is mixture of methylene chloride/methanol/dimethyl formamide.

23. One-pot process for the preparation of prasugrel compound of formula-1, 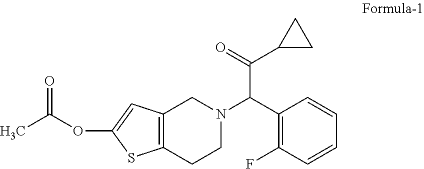
comprising:
a) Reacting the 5,6,7,7a-tetrahydro-4H-thieno[3,2-c]pyridine-2-one acid addition salt compound of formula-12 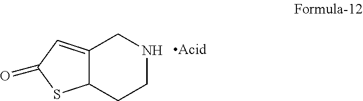
or its free base with a-cyclopropylcarbonyl-2-fluorobenzyl bromide compound of formula-3 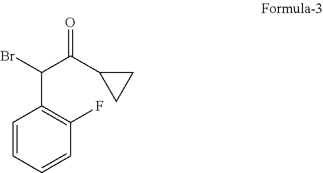
in presence of suitable organic base or inorganic base in a suitable solvent selected from nitrile solvent, ester solvent, polar aprotic solvent and ketone solvent or mixtures thereof,
b) filtering the unwanted solid and washing with suitable solvent,
c) treating the filtrate containing 5-(α-cyclopropylcarbonyl-2-fluorobenzyl)-2-oxo-2,4,5,6,7,7a-hexahydrothieno[3,2-c] pyridine compound of formula-7 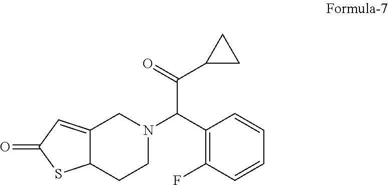
with acetic anhydride in presence of a suitable organic base,
d) extracting the reaction mixture with suitable hydrocarbon solvent,
e) distilling off the solvent completely under reduced pressure,
f) crystallizing the obtained residue from a suitable alcoholic solvent to provide the prasugrel compound of formula-1.

24. A process for the preparation of 1-cyclopropyl-2-(2-fluorophenyl) ethanone compound of formula-16, 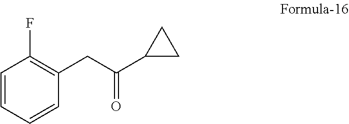
comprising:
Reacting the 2-(2-fluorophenyl) acetic acid compound of formula-13 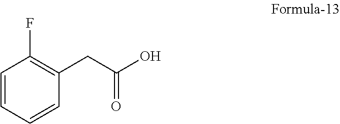
with N,O-dialkylhydroxylamine or its salts compound of general formula-14, 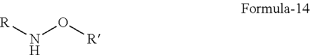
Wherein R and R′ each independently represents C₁₋₆ alkyl group, having a straight chain or branched chain,
in the presence of a base in a suitable solvent provides 2-(2-fluorophenyl)-N-alkoxy-N-alkylacetamide compound of general formula-15, 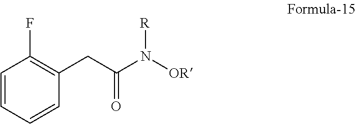
Wherein R and R′ each as defined above,
reacting the 2-(2-fluorophenyl)-N-alkoxy-N-alkylacetamide compound of general formula-15 with cyclopropyl magnesium bromide in a suitable solvent to provide 1-cyclopropyl-2-(2-fluorophenyl)ethanone compound of formula-16.

25. A process for the preparation of 1-cyclopropyl-2-(2-fluorophenyl) ethanone compound of formula-16, 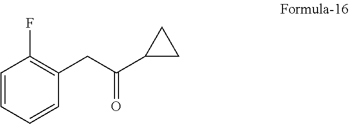
comprising:
Reacting the 2-(2-fluorophenyl)acetic acid compound of formula-13 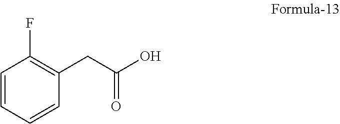
with N,O-dimethylhydroxylamine or its salts compound of formula-14a, 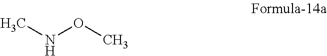
in the presence of dicyclohexylcarbodiimide (DCC) 1-hydroxybenzotriazole (HOBt) and in presence of triethyl amine in methylene chloride provides 2-(2-fluorophenyl)-N-methoxy-N-methylacetamide compound of formula-15a, 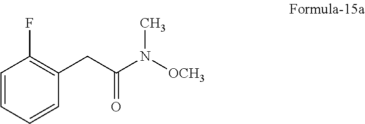
reacting the 2-(2-fluorophenyl)-N-methoxy-N-methylacetamide compound of formula-15a with cyclopropyl magnesium bromide in tetrahydrofuran to provide 1-cyclopropyl-2-(2-fluorophenyl)ethanone compound of formula-16.

26. A process for the purification of 1-cyclopropyl-2-(2-fluorophenyl) ethanone compound of formula-16, 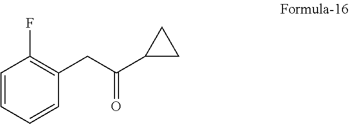
which comprises subjecting the crude 1-cyclopropyl-2-(2-fluorophenyl) ethanone to fractional distillation under reduced pressure to obtain pure 1-cyclopropyl-2-(2-fluorophenyl) ethanone.

27. The process according to claim 26, wherein the purification is carried out by fractional distillation and the pure compound fractions obtained at a vapour temperature of 80-90° C.

28. A process for the preparation of prasugrel or its pharmaceutically acceptable salt thereof containing less than 3% contaminant of methyl keto impurity comprising:
a) Preparing the 1-cyclopropyl-2-(2-fluorophenyl) ethanone compound of formula-16 by the process of claim 24, and
b) converting the compound of formula-16 into prasugrel or its pharmaceutically acceptable salts.

29. 1-cyclopropyl-2-(2-fluorophenyl) ethanone compound of formula-16 containing less than 4.0% of 1-methyl-2-(2-fluorophenyl)ethanone by GC, preferably less than 1.0% and more preferably less than 0.1% by GC.

30. Prasugrel or its pharmaceutically acceptable salts containing less than 3% of methyl keto impurity by HPLC, preferably less than 1.0% by HPLC and more preferably less than 0.1% by HPLC.

31. A process for the purification of prasugrel comprising recrystallizing the crude prasugrel from acetonitrile/isopropyl alcohol or a mixture of acetonitrile and isopropylalcohol.

32. 2-(2-fluorophenyl)-N-alkoxy-N-alkylacetamide compound represented by the following general formula-15 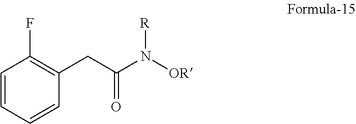
Wherein R and R′ each independently represents C₁₋₆ alkyl group, having a straight chain or branched chain.

33. Use of compound of formula-15 as claimed in claim 29 in the preparation of highly pure 1-cyclopropyl-2-(2-fluorophenyl) ethanone and prasugrel or its pharmaceutically acceptable salts.

34. A packing of Prasugrel hydrochloride to control desacetyl impurity and other impurities comprising:
a) Packing the prasugrel hydrochloride in clear low-density polyethylene bag and sealing the bag with vacuum sealer,
b) placing the above obtained bag in black color low-density polyethylene bag and seal the bag with vacuum sealer,
c) placing the above bag in triple laminated bag and sealing the bag with vacuum sealer,
d) placing the above bag in HDPE container and sealing the container, and
e) storing the container at control room temperature.

35. Prasugrel hydrochloride having mean particle size in the range of 40 to 80 microns and D₉₀ is in the range of 90 to 200 microns.

36. (canceled)

37. A process for the preparation of prasugrel or its pharmaceutically acceptable salt thereof containing less than 3% contaminant of methyl keto impurity, comprising:
c) Preparing the 1-cyclopropyl-2-(2-fluorophenyl) ethanone compound of formula-16 by the process of claim 26, and
d) converting the compound of formula-16 into prasugrel or its pharmaceutically acceptable salts.
